SEQUENCE LISTING (25 sequences)

SEQ ID NO: 1 - DNA, 1997 bp, Homo sapiens
  CDS (352)..(1608)
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - DNA, 2029 bp, Homo sapiens
  CDS (411)..(1475)
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 4 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 5 - DNA, 1997 bp, Homo sapiens
  misc_feature (817)..(819): n = any triplet that does not translate into a Cysteine or a stop codon
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccannngtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 6 - protein, 419 aa, Homo sapiens
  misc_feature (156)..(156): Xaa = is any amino acid other than Cysteine
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPXVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 7 - DNA, 468 bp, Homo sapiens
atgcacttgctgggcttcttctctgtggcgtgttctctgctcgccgctgcgctgctcccgggtcctcgcgaggcgcccgccgccgccgccgccacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgt

SEQ ID NO: 8 - protein, 156 aa, Homo sapiens
MHLLGFFSVACSLLAAALLPGPREAPAAAAATEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 9 - DNA, 402 bp, Homo sapiens
atgtacagagagtgggtagtggtgaatgttttcatgatgttgtacgtccagctggtgcagggctccagtaatgaattctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactca

SEQ ID NO: 10 - protein, 134 aa, Homo sapiens
MYREWVVVNVFMMLYVQLVQGSSNEFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYS

SEQ ID NO: 11 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 12 - DNA, 22 bp, Artificial sequence
  Synthetic primer
acattggtgtgcacctccaagc

SEQ ID NO: 13 - DNA, 27 bp, Artificial sequence
  Synthetic primer
aataatggaatgaacttgtctgtaaac

SEQ ID NO: 14 - DNA, 24 bp, Artificial sequence
  Synthetic primer
aaatcagttcgaggaaagggaaag

SEQ ID NO: 15 - DNA, 21 bp, Artificial sequence
  Synthetic primer
ccctgtgggccttgctcagag

SEQ ID NO: 16 - DNA, 35 bp, Artificial sequence
  Synthetic primer
ccatgctcgagagtctttcctggtgagagatctgg

SEQ ID NO: 17 - protein, 419 aa, Homo sapiens; identical to SEQ ID NO: 6
  misc_feature (156)..(156): Xaa = any or no amino acid

SEQ ID NO: 18 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 19 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 20 - protein, 317 aa, Homo sapiens [sequence omitted]

SEQ ID NO: 21 - DNA, 8815 bp, Homo sapiens
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacggctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacatccccagggaagatgtagactatcacctgtacccacacggtccgggactcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaacttgaagttcatctatttgatataagacaccttcgggggaaataattcctgtgaatattctttttcaattcagcaaacatttgaaaatctatgatgtgcaagtctaattgttgatttcagtacaagattttctaaatcagttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaataaaaatgactaagatattaaaaaaaaaaaaaaaaaa

SEQ ID NO: 22 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 23 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 24 - protein, 475 aa, Homo sapiens [sequence omitted]

SEQ ID NO: 25 - protein, 226 aa, Homo sapiens [sequence omitted]

(19) United States
(12) Patent Application Publication
(10) Pub. No.: US 2010/0041605 A1
(43) Pub. Date: Feb. 18, 2010

(54) Use of VEGF-C or VEGF-D in Reconstructive Surgery

(75) Inventors: Kari Alitalo, Helsinki (FI); Anne Saaristo, Helsinki (FI); Marika Karkkainen, Helsinki (FI); Tuomas Tammela, Helsinki (FI); Sirpa Asko-Seljavaara, Helsinki (FI); Seppo Yla-Herttuala, Vuorela (FI); Yulong He, Helsinki (FI)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: Vegenics Limited, Toorak (AU)

(21) Appl. No.: 12/490,037

(22) Filed: Jun. 23, 2009

Related U.S. Application Data
(63) Continuation of application No. 11/782,225, filed on Jul. 24, 2007, now abandoned.
Application No. 11/782,225 is a continuation of application No. 10/868,549, filed on Jun. 14, 2004, now abandoned.
(60) Provisional application No. 60/478,114, filed on Jun. 12, 2003.
(60) Provisional application No. 60/478,390, filed on Jun. 12, 2003.

(51) Int. Cl. A61K 38/18; A61K 9/70; A61P 17/00; A61P 17/02

(52) U.S. Cl. 514/12; 424/443

(57) ABSTRACT

The present invention provides materials and methods for repairing tissue and using vascular endothelial growth factor C (VEGF-C) genes and/or proteins. Methods and materials related to the use of VEGF-C for the reduction of edema and improvement of skin perfusion is provided. Also provided is are materials and methods for using VEGF-C before, during, and after reconstructive surgery.

The present application claims the benefit of priority of U.S. Provisional Patent Application No. 60/478,114, filed Jun. 12, 2003. The present application also claims the benefit of priority of U.S. Patent Application No. 60/478,390, filed Jun. 12, 2003. The entire text of each of the foregoing application is specifically incorporated herein by reference.

### FIELD OF THE INVENTION

The present invention generally relates to materials and methods to improve healing of skin and underlying tissue following a surgical procedure.

### BACKGROUND OF THE INVENTION

Skin flap survival following surgical procedures, especially reconstructive surgical procedures, is often compromised by, among other complications, infection, ischemia and tissue edema. Tissue and skin flap breakdown remain a major problem in plastic surgery, especially in patients suffering from diabetic microangiopathy or other forms of peripheral vascular disease. In such patients wound healing is often delayed and defective and in these patients complications may lead to necrosis and eventually require costly and painful secondary surgical procedures.

The vascular endothelial growth factor (VEGF) family currently includes six members, which are important regulators of angiogenesis and lymphangiogenesis: VEGF, placenta growth factor (P1GF), VEGF-B, VEGF-C, VEGF-D, and VEGF-E (Li, X., et al., Int. J. Biochem. Cell Biol., 33:421-426(2001)). VEGF is also known as vascular permeability factor, and it is more potent than histamine in increasing capillary permeability to plasma proteins (Li, X., et al., supra). VEGF binds selectively and with high affinity to receptor tyrosine kinases VEGFR-1 and VEGFR-2 (Li, X., et al., supra). Angiopoietins (Angs) constitute another family of endothelial growth factors that are ligands for the endothelium-specific receptor tyrosine kinase, Tie-2 (Tek) (Davis, S., et al., Cell, 87:1161-1169 (1996)). Although Angs do not appear to induce new vessel growth, they may be involved in vessel stabilization. Vascular permeability induced by VEGF, for example, is reported to be blocked by angiopoietin-1 (Ang-1) (Thurston, G., et al., Nat. Med., 6:460-462 (2000)).

VEGF has been employed as a growth factor candidate in treatments aimed at increasing blood supply and tissue perfusion in compromised tissues. (Padubidri, A., et al., Ann. Plast. Surg., 37:604 (1996)). Recent reports have focused on VEGF gene therapies in order to generate a more efficient and sustained response than protein therapy (Faries, P. L., et al., Ann. Vasc. Surg., 14:181-188 (2000)).

Although VEGF is a potent inducer of angiogenesis, the vessels that it helps to create are immature, tortuous, and leaky, often lacking perivascular support structures (Carmeliet, P., Nat. Med. 6:1102-1103 (2000); Blau, H. M., et al., Nat. Med., 7:532-534 (2001); Epstein, S. E., et al., Circulation, 104:115-119 (2001)). Only a fraction of the blood vessels induced in response to VEGF in the dermis and in subcutaneous fat tissue were stabilized and functional after adenoviral treatment of the skin of nude mice (Pettersson, A., et al., Lab. Invest., 80:99-115 (2000); Sundberg, C., et al., Am. J. Pathol., 158:1145-1160 (2001)), while intramuscular vessels developed into a hemangioma-like proliferation or regressed with resulting scar tissue (Pettersson, A., et al., supra; Springer, M. L., et al., Mol. Cell., 2:549-558 (1998)). Furthermore, edema induced by VEGF overexpression complicates VEGF-mediated neovascularization, although two reports suggests that it can be reduced by co-administering Ang-1 for vessel stabilization (Thurston, G., et al., Science., 286:2511-2514 (1999); Thurston, G., et al., Nat. Med., 6:460-462 (2000)).

While the aforementioned VEGF-based therapies have shown some promise with respect to the development of new blood vessels, there remains a need for the development of improved therapeutic approaches for surgical procedures involving skin flap or skin graft attachment that reduce the edema, skin perfusion, necrosis, and other problems associated with skin healing.

### SUMMARY OF THE INVENTION

The present invention addresses long-felt needs in the field of medicine by providing materials and methods to improve healing of skin and/or underlying tissue following a surgical procedure. Improved healing may be indicated by a variety of criteria, including reduced swelling/edema; and/or reduced infections; and/or reduced tissue breakdown, necrosis, or ischemia; and/or increased tissue perfusion; and/or reduced pain; and/or reduced scarring; and/or more rapid healing, for example. The esthetic outcome of the operations may heavily depend on the restoration of the normal tissue and vessel architecture.

For example, the invention provides a method of improving the healing of a skin graft or skin flap to underlying tissue of a mammalian subject, comprising contacting skin graft or skin flap tissue or underlying tissue with a composition comprising a healing agent selected from the group consisting of Vascular Endothelial Growth Factor C (VEGF-C) polynucleotides, VEGF-C polypeptides, Vascular Endothelial Growth Factor D (VEGF-D) polynucleotides, and VEGF-D polypeptides. In a preferred embodiment, the healing agent is present in the composition in an amount effective to reduce edema or increase perfusion at the skin graft or skin flap, thereby improving the healing of the skin graft or skin flap.

In another preferred embodiment, the mammalian subject is a human. In another preferred embodiment, the mammalian subject is diabetic.

In the context of contacting skin flap or skin graft tissue cell with a composition, the term “contacting” is intended to include administering the composition to a subject such that the composition physically touches cells of the skin graft, skin flap tissue, or underlying tissue to permit the healing agent to exert its biological effects on such cells. The contacting may occur in vivo, where the composition is administered to the subject or applied to the skin flap tissue or underlying tissue (i.e., tissue of the mammalian subject to which the skin flap or skin graft will be attached). “Contacting” may also include incubating the composition and cells or graft tissue together in vitro (e.g., adding the composition to cells in culture or applying or injecting it into graft tissue that is not yet physically attached to the subject).

The term “VEGF-C polypeptide” includes any polypeptide that has a VEGF-C or VEGF-C analog amino acid sequence (as defined elsewhere herein in greater detail) and that possesses VEGFR-3 binding and stimulatory properties. The term “VEGF-C polynucleotide” includes any polynucleotide (e.g., DNA or RNA, single- or double-stranded) comprising a nucleotide sequence that encodes a VEGF-C polypeptide. Due to the well-known degeneracy of the genetic code, multiple VEGF-C polynucleotide sequences encode any selected VEGF-C polypeptide.

In a preferred embodiment, the method further includes a step of attaching the skin graft of skin flap to the underlying tissue. In one variation, the contacting precedes the attaching. Alternatively, the contacting occurs subsequent to the attaching. In a preferred variation, the attaching step includes surgical connection of blood vessels between the underlying tissue and the skin graft or skin flap.

As described below in greater detail, the improvements to surgical skin graft/skin flap procedures described herein are applicable to a wide variety of surgeries. For example, in one embodiment, the invention provides a method of improving the healing of a skin graft or skin flap to underlying tissue of a subject wherein the underlying tissue is breast tissue. In a preferred embodiment, the skin graft or skin flap is attached in a breast augmentation, breast reduction, mastopexy, or gynecomastia procedure.

Still another embodiment of the invention provides a method of improving the healing of a skin graft or skin flap to underlying tissue of a mammalian subject wherein the skin graft or skin flap is attached in a cosmetic surgery procedure. In a preferred embodiment, the cosmetic surgery is a facial cosmetic surgery procedure selected from the group consisting of rhytidectomy, browlift, otoplasty, blepharoplasty, rhinoplasty, facial implant, and hair replacement therapy.

In still another embodiment of the invention provides a method of improving the healing of a skin graft or skin flap to underlying tissue of a mammalian subject wherein the skin graft or skin flap is attached in an abdominoplasty (abdominal lipectomy) or liposuction procedure.

Another embodiment of the invention provides a method of improving the healing of a skin graft or skin flap to underlying tissue of a mammalian subject wherein the skin graft or skin flap is attached in a reconstructive surgery. In a preferred embodiment, the reconstructive surgery corrects a congenital defect selected from the group consisting of birthmark, cleft palate, cleft lip, syndactyly, urogenital and anorectal malformations, craniofacial birth defects, ear and nasal deformities, and vaginal agenesis. In another preferred embodiment, the reconstructive surgery corrects a defect from an injury, infection, or disease. In another preferred embodiment, the reconstructive surgery corrects damage from a burn or skin cancer (or skin cancer related treatment). In another preferred embodiment, the reconstructive surgery is breast reconstruction following mastectomy or injury.

The materials and methods of the invention may be practiced with a skin graft that is a split thickness, full thickness, or composite graft, and/or a skin flap that is a local flap, a regional flap, a musculocutaneous flap, an osteomyocutaneous flap and/or a soft tissue flap. One can also contemplate the use of in vitro epidermal keratinocyte cultures and epidermal sheets formed therefrom into which the VEGF-C polynucleotides have been transfected. The epidermal sheets are administered to a patient, for example, to promote re-epthelialization of burn wounds.

Multiple healing agents are contemplated to be used, alone or in combination, to practice the present invention. In one embodiment, the healing agent comprises a VEGF-C polynucleotide that encodes a VEGF-C polypeptide. In a preferred embodiment, the VEGF-C polynucleotide further encodes a heparin-binding domain in frame with the VEGF-C polypeptide. In a related embodiment, the VEGF-C polypeptide comprises the formula X—B—Z or Z—B—X, wherein X binds Vascular Endothelial Growth Factor Receptor 3 (VEGFR-3) and comprises an amino acid sequence at least 90%, identical to a VEGFR-3 ligand selected from the group consisting of (a) the prepro-VEGF-C amino acid sequence set forth in SEQ ID NO: 2; and (b) fragments of (a) that bind VEGFR-3; wherein Z comprises a heparin-binding amino acid sequence; and wherein B comprises a covalent attachment linking X to Z.

In another embodiment, the healing agent comprises a polypeptide which comprises an amino acid sequence at least 80%, and more preferably at least 90%, 95%, 97%, 98%, or 99% identical to the amino acid sequence set forth in SEQ ID NO: 2 or to a fragment thereof that binds VEGFR-3, where the polypeptide binds to VEGFR-3.

In still another embodiment, the aforementioned method is provided wherein the healing agent comprises a VEGF-D polynucleotide that encodes a VEGF-D polypeptide. In a related embodiment, the VEGF-D polypeptide comprises an amino acid sequence at least at least 80%, and more preferably at least 90%, 95%, 97%, 98%, or 99% identical to the amino acid sequence set forth in SEQ ID NO: 4 or to a fragment thereof that is effective to bind VEGFR-3, wherein the polypeptide binds to VEGFR-3. In yet another related embodiment, the healing agent comprises a VEGF-D polypeptide. In another embodiment, the VEGF-D polypeptide comprises the amino acid sequence set forth in SEQ ID NO: 4 or a fragment thereof that binds VEGFR-3.

In preferred embodiments, the VEGF-C polynucleotide further comprises additional sequences to facilitate the VEGF-C gene therapy. In a preferred embodiment, the polynucleotide further comprises a nucleotide sequence encoding a secretory signal peptide, wherein the sequence encoding the secretory signal peptide is connected in-frame with the sequence that encodes the VEGF-C polypeptide. In a preferred embodiment, the polynucleotide further comprises a promoter and/or enhancer sequence operably connected to the sequence that encodes the secretory signal sequence and VEGF-C polypeptide, wherein the promoter sequence promotes transcription of the sequence that encodes the secretory signal sequence and the VEGF-C polypeptide in cells of the mammalian subject. In one variation, the promoter is a constitutive promoter that promotes expression in a variety of cell types, such as the cytomegalovirus promoter/enhancer (Lehner et al., J. Clin. Microbiol., 29:2494-2502 (1991); Boshart et al., Cell, 41:521-530 (1985)); or Rous sarcoma virus promoter (Davis et al., Hum. Gene Ther., 4:151 (1993)) or simian virus 40 promoter. Also contemplated is an endothelial cell specific promoter such as Tie promoter (Korhonen et al., Blood, 86(5): 1828-1835 (1995); U.S. Pat. No. 5,877,020). In a highly preferred embodiment, the promoter sequence comprises a skin specific promoter. Preferred promoter sequences include the K14, K5, K6, K16 promoters for the epidermis and alpha 1(I) collagen promoter for the dermis (Diamond, I., et al., J. Invest. Dermatol., 115(5):788-794 (2000); Galera, P., et al., Proc. Natl. Acad. Sci. USA, 91(20):9372-9376 (1994); Wawersik, M. J., et al., Mol. Biol. Cell, 12(11):3439-3450 (2001)). All of the foregoing documents are incorporated herein by reference in the entirety. In another preferred embodiment, the polynucleotide further comprises a polyadenylation sequence operably connected to the sequence that encodes the VEGF-C polypeptide.

Irrespective of which VEGF-C polypeptide is chosen, the VEGF-C polynucleotide preferably comprises a nucleotide sequence encoding a secretory signal peptide fused in-frame with the VEGF-C polypeptide sequence. The secretory signal peptide directs secretion of the VEGF-C polypeptide by the cells that express the polynucleotide, and is cleaved by the cell from the secreted VEGF-C polypeptide. For example, the VEGF-C polynucleotide could encode the complete prepro-VEGF-C sequence set forth in SEQ ID NO: 2 (which includes natural VEGF-C signal peptide); or could encode the VEGF-C signal peptide fused in-frame to a sequence encoding a recombinantly-processed VEGF-C (e.g., amino acids 103-227 of SEQ ID NO: 2) or VEGF-C analog. Moreover, there is no requirement that the signal peptide be derived from VEGF-C. The signal peptide sequence can be that of another secreted protein, or can be a completely synthetic signal sequence effective to direct secretion in cells of the mammalian subject.

In one embodiment, the VEGF-C polynucleotide of the invention comprises a nucleotide sequence that will hybridize to a polynucleotide that is complementary to the human VEGF cDNA sequence specified in SEQ ID NO: 1 under the following exemplary stringent hybridization conditions: Hybridization at 42° C. in 50% formamide, 5×SSC, 20 mM Na.PO₄, pH 6.8; and washing in 1×SSC at 55° C. for 30 minutes; and wherein the nucleotide sequence encodes a polypeptide that binds and stimulates human VEGFR-2 and/or VEGFR-3. It is understood that variation in these exemplary conditions can be made based on the length and GC nucleotide content of the sequences to be hybridized. Formulas standard in the art are appropriate for determining appropriate hybridization conditions. See Sambrook et al., Molecular Cloning: A Laboratory Manual (Second ed., Cold Spring Harbor Laboratory Press, 1989) §§9.47-9.51.

The polynucleotide may further optionally comprise sequences whose only intended function is to facilitate large-scale production of the vector, e.g., in bacteria, such as a bacterial origin of replication and a sequence encoding a selectable marker. However, in a preferred embodiment, such extraneous sequences are at least partially cleaved off prior to administration to humans according to methods of the invention.

In one embodiment, a “naked” VEGF-C transgene (i.e., a transgene without a viral, liposomal, or other vector to facilitate transfection) is employed for gene therapy. In this embodiment, the VEGF-C polynucleotide preferably comprises a suitable promoter and/or enhancer sequence for expression in the target mammalian cells, the promoter being operatively linked upstream (i.e., 5′) of the VEGF-C coding sequence. The VEGF-C polynucleotide also preferably further includes a suitable polyadenylation sequence (e.g., the SV40 or human growth hormone gene polyadenylation sequence) operably linked downstream (i.e., 3′) of the VEGF-C coding sequence.

Polynucleotide healing agents preferably are incorporated into a vector to facilitate delivery to target cells in the mammalian host cells, and a variety of vectors can be employed. Thus, in one embodiment, the invention provides a method of improving the healing of a skin graft or skin flap to underlying tissue of a subject wherein the healing agent comprises a gene therapy vector that comprises the VEGF-C polynucleotide. In a preferred embodiment, the gene therapy vector is an adenoviral or adeno-associated viral vector. In a highly preferred embodiment, the vector comprises a replication-deficient adenovirus, the adenovirus comprising the polynucleotide operably connected to a promoter and flanked by adenoviral polynucleotide sequences. The adenoviral vector should be included in the composition at a titer conducive to promoting healing according to the invention. In an embodiment where the VEGF-C transgene is administered in an adenovirus vector, the vector is preferably administered in a pharmaceutically acceptable carrier at a titer of 10⁷-10¹³ viral particles, and more preferably at a titer of 10⁹-10¹¹ viral particles. The adenoviral vector composition preferably is infused over a period of 15 seconds to 30 minutes, more preferably 1 to 10 minutes.

The invention is not limited to a particular vector because a variety of vectors are suitable to introduce the VEGF-C transgene into the host. Exemplary vectors that have been described in the literature include replication-deficient retroviral vectors, including but not limited to lentivirus vectors (Kim et al., J. Virol., 72(1): 811-816 (1998); Kingsman & Johnson, Scrip Magazine, October, 1998, pp. 43-46.); adeno-associated viral vectors (Gnatenko et al., J. Investig. Med., 45: 87-98 (1997)); adenoviral vectors (See, e.g., U.S. Pat. No. 5,792,453; Quantin et al., Proc. Natl. Acad. Sci. USA, 89: 2581-2584 (1992); Stratford-Perricadet et al., J. Clin. Invest., 90: 626-630 (1992); and Rosenfeld et al., Cell, 68: 143-155 (1992)); Lipofectin-mediated gene transfer (BRL); liposomal vectors (See, e.g., U.S. Pat. No. 5,631,237 (Liposomes comprising Sendai virus proteins)); and combinations thereof. Additionally, the VEGF-C transgene can be transferred via particle-mediated gene transfer (Gurunluonglu, R., et al., Ann. Plast. Surg., 49:161-169 (2002)). All of the foregoing documents are incorporated herein by reference in the entirety.

In embodiments employing a viral vector, preferred polynucleotides include a suitable promoter and polyadenylation sequence as described herein. The polynucleotide further includes vector polynucleotide sequences (e.g., adenoviral polynucleotide sequences) operably connected to the sequence encoding a VEGF-C polypeptide.

Thus, in one embodiment, the composition to be administered comprises a vector, wherein the vector comprises the VEGF-C polynucleotide. In a preferred embodiment, the vector is an adenovirus vector. In a highly preferred embodiment, the adenovirus vector is replication-deficient, i.e., it cannot replicate in the mammalian subject due to deletion of essential viral-replication sequences from the adenoviral genome. For example, the inventors contemplate a method wherein the vector comprises a replication-deficient adenovirus, the adenovirus comprising the VEGF-C polynucleotide operably connected to a promoter and flanked on either end by adenoviral polynucleotide sequences.

In one embodiment, the healing agent comprises a VEGF-C polypeptide. In a preferred embodiment, the VEGF-C polypeptide comprises a mammalian VEGF-C polypeptide. In a highly preferred embodiment, especially for treatment of humans, the VEGF-C polypeptide comprises a human VEGF-C polypeptide. By “human VEGF-C” is meant a polypeptide corresponding to a naturally occurring protein (prepro-protein, partially-processed protein, or fully-processed mature protein) encoded by any allele of the human VEGF-C gene, or a polypeptide comprising a biologically active fragment of a naturally-occurring mature protein. For example, the VEGF-C polypeptide comprises the amino acid sequence set forth in SEQ ID NO: 2 or comprises a fragment thereof that binds to VEGFR-2 and VEGFR-3 and stimulates VEGFR-2 and VEGFR-3 phosphorylation in cells that express these receptors.

A polypeptide comprising amino acids 103-227 of SEQ ID NO: 2 is specifically contemplated. For example, polypeptides having an amino acid sequence comprising a continuous portion of SEQ ID NO: 2, the continuous portion having, as its amino terminus, an amino acid selected from the group consisting of positions 32-111 of SEQ ID NO: 2, and having, as its carboxyl terminus, an amino acid selected from the group consisting of positions 228-419 of SEQ ID NO: 2 are contemplated. As explained elsewhere herein in greater detail, VEGF-C biological activities, especially those mediated through VEGFR-2, increase upon processing of both an amino-terminal and carboxyl-terminal pro-peptide. Thus, an amino terminus selected from the group consisting of positions 102-131 of SEQ ID NO: 2 is preferred, and an amino terminus selected from the group consisting of positions 103-111 of SEQ ID NO: 2 is highly preferred. Likewise, a carboxyl terminus selected from the group consisting of positions 215-227 of SEQ ID NO: 2 is preferred. The term “human VEGF-C” also is intended to encompass polypeptides encoded by allelic variants of the human VEGF-C characterized by the sequences set forth in SEQ ID NOs: 1 & 2.

Moreover, it is within the capabilities of the person skilled in the art to make and use analogs of human VEGF-C (and polynucleotides that encode such analogs) wherein one or more amino acids have been added, deleted, or replaced with other amino acids, especially with conservative replacements, and wherein the receptor binding and stimulating biological activity has been retained. Analogs that retain VEGFR-3 binding and stimulating VEGF-C biological activity are contemplated as VEGF-C polypeptides for use in the present invention. In a preferred embodiment, analogs having 1, 2, 3, 4, 5, 6, 7, 8, 9, 10, 11, 12, 13, 14, 15, 16, 17, 18, 19, 20, 21, 22, 23, 24, or 25 such modifications and that retain VEGFR-3 binding and stimulating VEGF-C biological activity are contemplated as VEGF-C polypeptides for use in the present invention. Polynucleotides encoding such analogs are generated using conventional PCR, site-directed mutagenesis, and chemical synthesis techniques.

In another preferred embodiment the VEGF-C polypeptide selectively binds VEGFR-3. By “selectively binds VEGFR-3” is meant that the polypeptide fails to significantly bind VEGFR-2 and is not proteolytically processed in vivo into a form that shows significant reactivity with VEGFR-2. An exemplary VEGFR-3 specific VEGF-C polypeptide comprises a VEGF-C156X polypeptide (See SEQ ID NO: 6 and corresponding nucleotide sequence in SEQ ID NO: 5), in which the cysteine at position 156 is replaced with an amino acid, X, other than cysteine (for example, serine; VEGF-C156S). By “VEGF-C156X polypeptide” is meant an analog wherein the cysteine at position 156 of SEQ ID NO: 2 has been deleted or replaced by another amino acid. A VEGF-C156X polypeptide analog can be made from any VEGF-C polypeptide of the invention that comprises all of SEQ ID NO: 2 or a portion thereof that includes position 156 of SEQ ID NO: 2. Preferably, the VEGF-C156X polypeptide analog comprises a portion of SEQ ID NO: 2 effective to permit binding to VEGFR-3 and has reduced VEGFR-2 binding affinity.

Also contemplated as VEGF-C polypeptides are non-human mammalian or avian VEGF-C polypeptides and polynucleotides. By “mammalian VEGF-C” is meant a polypeptide corresponding to a naturally occurring protein (prepro-protein, partially-processed protein, or fully-processed mature protein) encoded by any allele of a VEGF-C gene of any mammal, or a polypeptide comprising a biologically active fragment of a mature protein.

In one embodiment of the method of the invention, the contacting and attaching are performed without use of an angiogenic polypeptide that binds VEGFR-1 or VEGFR-2.

In another embodiment, the method includes contacting the skin graft or skin flap or underlying tissue with an angiogenic growth factor that promotes blood vessel growth. For example, the method comprises contacting the skin graft or skin flap or underlying tissue with a composition comprising VEGF-C, VEGF-C156S and/or VEGF-D polynucleotide or polypeptide in combination with a VEGF, VEGF-B, VEGF-E, P1GF, Ang-1, EGF, PDGF-A, PDGF-B, PDGF-C, PDGF-D, FGF, TGF-β, and/or IGF, polynucleotide or polypeptide. In a preferred embodiment, the angiogenic growth factor is substantially free of vascular permeability increasing activity.

As an alternative to being included in a pharmaceutical composition of the invention including a first protein, a second protein or a therapeutic agent may be concurrently administered with the first protein (e.g., at the same time, or at differing times provided that therapeutic concentrations of the combination of agents is achieved at the treatment site).

The composition(s) used to practice methods of the invention optionally comprise additional materials besides the healing agent. For example, the composition preferably includes a pharmaceutically acceptable carrier.

In a highly preferred embodiment, the composition is administered locally, e.g., to the site of the skin graft or flap. In one variation of the method, the contacting step comprises injecting the composition intradermally or subdermally. In another variation, the contacting comprises injection of the composition into the dermis of the skin graft or skin flap. In still another variation, the mode of contacting comprises topical application of the composition to the skin graft or skin flap. Topical application can be achieved by a variety of materials and techniques, including use of ointments, creams, lotions, transdermal delivery patches, and composition applied to wound dressings.

In still another variation, the contacting is achieved by applying/impregnating sutures with the composition and using the sutures to attach the skin flap/graft to the underlying tissue. For example, intracutaneous resorbable continuous (zigzag) suture is immersed in the composition and used to attach the flap. Vessels should grow to the site of the resorbable suture.

In still another variation, endothelial cells, endothelial progenitor cells, smooth muscle cells, or keratinocytes are transfected ex vivo with the VEGF-C transgene, and the transfected cells are administered to the mammalian subject. Also keratinocytes can be transfected (with VEGF-C transgene) in vitro and then administered to the subject. VEGF-C released in vivo from the transfected cells would then attract the endothelial cells on which the VEGF-C receptors are expressed to migrate and make new vessels. Exemplary procedures for seeding a vascular graft with genetically modified endothelial cells are described in U.S. Pat. No. 5,785,965, incorporated herein by reference.

If the mammalian subject is receiving a vascular graft with the skin graft, the VEGF-C transgene-containing composition may be directly applied to the isolated vessel segment prior to its being grafted in vivo.

Administration via one or more intravenous injections subsequent to the surgical procedure also is contemplated. Localization of the VEGF-C polypeptides to the site of the procedure occurs due to expression of VEGF-C receptors on proliferating endothelial cells. Localization is further facilitated by recombinantly expressing the VEGF-C as a fusion polypeptide (e.g., fused to an apolipoprotein B-100 oligopeptide as described in Shih et al., Proc. Nat'l. Acad. Sci. USA, 87:1436-1440 (1990)). Co-administration of VEGF-C polynucleotides and VEGF-C polypeptides also is contemplated.

Still other healing agents besides VEGF-C polypeptide and polynucleotides are contemplated to be used with methods of the present invention. In one embodiment, the healing agent comprises a VEGF-D polypeptide or a polynucleotide that encodes a VEGF-D polypeptide. Such methods are practiced essentially as described herein with respect to VEGF-C-encoding polynucleotides or polypeptides, except that VEGF-D polynucleotides or polypeptides are employed. Thus, for example, the description above relating to the use of promoter sequences, vectors, and the like is equally applicable to VEGF-D polynucleotides. A detailed description of the human VEGF-D gene and protein are provided in Achen, et al., Proc. Nat'l Acad. Sci. U.S.A., 95(2):548-553 (1998); International Patent Publication No. WO 98/07832, published 26 Feb. 1998; and in Genbank Accession No. AJ000185, all incorporated herein by reference. A cDNA and deduced amino acid sequence for prepro-VEGF-D is set forth herein in SEQ ID NOs: 3 and 4. Due to the well-known degeneracy of the genetic code, there exist multiple VEGF-D encoding polynucleotide sequences for any VEGF-D polypeptide, any of which may be employed according to the methods taught herein.

As described herein in detail with respect to VEGF-C, the use of polynucleotides that encode VEGF-D fragments, VEGF-D analogs, VEGF-D allelic and interspecies variants, and the like which bind and stimulate phosphorylation of VEGFR-3 are all contemplated as being encompassed by the present invention.

Moreover, a treatment regimen comprising the simultaneous administration of VEGF-C protein (to provide immediate therapy to the target vessel) with a VEGF-C transgene (to provide sustained therapy for several days or weeks) is specifically contemplated as a variation of the invention.

In another variation, the VEGF-C or VEGF-D is covalently linked to another peptide that modulates localization or biological activity. This is preferably achieved at the polynucleotide level. For example, a polynucleotide sequence that encodes the VEGF-C or VEGF-D growth factor domain is covalently fused to a nucleotide sequence encoding an amino acid sequence that directs the recombinant growth factor distribution to target tissues. For example, a sequence is linked that will influence new vessels to grow along collagenous bundles or on the surface of basal laminae. It is contemplated that numerous protein domains such as collagen or other extracellular matrix binding domains/sequences could be used to direct the distribution of the recombinant growth factor.

Additional domains have been described in laminin, which interacts with basal lamina proteins and so on (Ries, A., et al., Eur. J. Biochem., 268(19):5119-5128 (2001); Salmivirta, K., et al., Exp. Cell Res., 279(2):188-201 (2002); Stetefeld, J., et al., Nat. Struct. Biol., 8(8):705-709 (2001)).

In one embodiment, the heparin-binding domain of VEGF or another heparin-binding growth factor is fused to the growth factor domain of VEGF-C. The heparin-binding domain of VEGF fused with the VEGF-C growth factor domain would result in slow release of the VEGF-C growth factor from heparin, similar to what has been described with VEGF165 (Keck, R. G., et al., Arch. Biochem. Biophys., 344:103-113 (1997); Fairbrother, W. J., et al., Structure, 6:637-648 (1998).

Heparin binding forms of VEGF-C and VEGF-D are described in greater detail in commonly owned, U.S. patent application Ser. No. ______ (Attorney Docket No. 28967/39359A, co-filed on Jun. 14, 2004) and U.S. Patent Application No. 60/478,390, filed Jun. 12, 2003, incorporated herein by reference.

In a related aspect, the invention provides materials and devices for practice of the above-described methods.

For example, further aspects of the invention are materials that are useful for improving the healing of a skin flap or skin graft to underlying tissue. For example, the invention provides the use of a VEGF-C polynucleotide, and/or a VEGF-C polypeptide and/or a VEGF-D polynucleotide and/or a VEGF-D polypeptide for the manufacture of a medicament to improve the healing of a skin flap or skin graft to underlying tissue. Such compositions are summarized above in the discussion of methods of the invention and described in further detail below. In addition to the aforementioned healing agent(s), the composition preferably further includes one or more pharmaceutically acceptable diluents, adjuvants, or carrier substances.

The polynucleotides, polypeptides, vectors, compositions, and the like that are described for use in methods of the invention are themselves intended as aspects of the invention.

The compositions are also presently valuable for veterinary applications. Particularly domestic animals and thoroughbred horses, in addition to humans, are desired patients for such treatment with a composition of the present invention.

Likewise, the invention also provides surgical devices that are used to reduce edema or increase perfusion at the skin graft or skin flap comprising a VEGF-C polynucleotide, a VEGF-C polypeptide, a VEGF-D polynucleotide, and/or a VEGF-D polypeptide.

For example, in one embodiment, the invention provides a transdermal patch for the administration of a composition of the invention, wherein the patch comprises a composition comprising a VEGF-C polynucleotide, a VEGF-C polypeptide, a VEGF-D polynucleotide, and/or a VEGF-D polypeptide. The thickness of the transdermal patch depends on the therapeutic requirements and may be adapted accordingly. Transdermal patches represent an alternative to the liquid forms of application. These devices can come in a variety of forms, all having the capability of adhering to the skin, and thereby permitting prolonged contact between the therapeutic composition and the target area. They also have the advantage of being relatively compact and portable, and permitting very precise delivery of a composition to the area to be treated. These patches come in a variety of forms, some containing fluid reservoirs for the active component, others containing dry ingredients that are released upon contact with moisture in the skin. Many require some form of adhesive to retain them in connection with the skin for an adequate period. A different type of patch is applied dry, with water applied to wet the patch to form a sticky film that is retained on the skin

As used herein “patch” comprises at least a topical composition according to the invention and a covering layer, such that, the patch can be placed over a surgically closed wound, incision, skin flap, skin graft, or burn, thereby positioning the patch/composition adjacent to the compromised tissue surface. Preferably, the patch is designed to maximize composition delivery through the stratum corneum, upper epidermis, and into the dermis, and to minimize absorption into the circulatory system, reduce lag time, promote uniform absorption, and reduce mechanical rub-off.

Preferred patches include (1) the matrix type patch; (2) the reservoir type patch; (3) the multi-laminate drug-in-adhesive type patch; and (4) the monolithic drug-in-adhesive type patch; (Ghosh, T. K., et al., Transdermal and Topical Drug Delivery Systems, Interpharm Press, Inc. p. 249-297 (1997) incorporated herein by reference). These patches are well known in the art and generally available commercially.

In another embodiment, the inventions provides a dressing for the delivery of a composition of the invention, wherein the dressing comprises a composition comprising a VEGF-C polynucleotide, a VEGF-C polypeptide, a VEGF-D polynucleotide, and/or a VEGF-D polypeptide. After application of the topical composition to the compromised tissue, the tissue may be covered with a dressing. The term “dressing”, as used herein, means a covering designed to protect and or deliver a (previously applied) composition. “Dressing” includes coverings such as a bandage, which may be porous or non-porous and various inert coverings, e.g., a plastic film wrap or other non-absorbent film. The term “dressing” also encompasses non-woven or woven coverings, particularly elastomeric coverings, which allow for heat and vapor transport. These dressings allow for cooling of the pain site, which provides for greater comfort.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. Where protein therapy is described, embodiments involving polynucleotide therapy (using polynucleotides that encode the protein) are specifically contemplated, and the reverse also is true. Where embodiments of the invention are described with respect to VEGF-C, it should be appreciated that analogous embodiments involving VEGF-D are specifically contemplated, including descriptions of how to make variants of wildtype molecules.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE DRAWING

FIG. 1 is a schematic depiction of a patch for the delivery of therapeutic compositions.

FIG. 2 shows that AdVEGF-C or AdVEGF-C156S induced a significant increase in the number of VEGFR-3 and PECAM-1 positive vessels relative to the AdLacZ control.

FIG. 3A schematically depicts the proteolytic processing of VEGF-C (Joukov et al., EMBO J 16: 3898-911, 1997). SS, signal sequence; N-term and C-term, N-terminal and C-terminal (silk homology domain) propeptides; VHD, VEGF homology domain; arrowheads, cleavage sites; and disulfide bonds are marked as —S—S— and dotted lines as non-covalent bonds.

FIG. 3B schematically depicts VEGF splice variants (named VEGF121, VEGF145, VEGF165, VEGF189, and VEGF206) generated by alternative splicing of the eight exons (numbered 1 to 8 shown at the bottom) of the human VEGF gene.

FIG. 3C is a schematic illustration of two VEGF-C/VEGF chimeric molecules comprised of the signal sequence and the VEGF homology domain of VEGF-C, and VEGF exon 6-8 or exon 7-8 encoded sequences (CA89 and CA65, respectively).

FIG. 3D is an autoradiogram depicting immunoprecipitation analysis of radiolabeled, secreted proteins in the conditioned medium from the 293T cells transfected with pEBS7/CA89, pEBS7/CA65 or the pEBS7 vector alone.

FIG. 4 is a graph depicting absorbance measurements (540 nm wavelength) of reaction products in a cell viability assay to measure biological activity of the chimeric molecules depicted in FIGS. 1C-1D. The biological activity of the VEGF-C chimeric proteins was demonstrated by a bioassay using Ba/F3 cells expressing a chimeric VEGFR-3/erythropoietin (Epo) receptor which transmitted survival and proliferation signals of VEGF-C for the IL-3 dependent Ba/F3/VEGFR-3 cells. Data represent the mean values from triplicate assays.

FIG. 5A. Immunoprecipitation and polyacrylamide gel electrophoresis of secreted proteins (labeled with 35S) from the conditioned medium of 293T cells transfected with pEBS7/CA89 (CA89), pEBS7/CA65 (CA65), pEBS7/VEGF-C N C (N C), or the pEBS7 vector, with neuropilin-1-Ig (NP1) and neuropilin-2-Ig (NP2)

FIG. 5B. Immunoprecipitation and polyacrylamide gel electrophoresis of secreted proteins (labeled with 35S) from the conditioned medium of 293T cells transfected with pEBS7/CA89 (CA89), pEBS7/CA65 (CA65), pEBS7/VEGF-CΔNΔC (NΔC), or the pEBS7 vector, with, and VEGFR-1-Ig (R-1), VEGFR-2-Ig (R-2) and VEGFR-3-Ig (R-3).

FIG. 6A. Analysis of viral expression of the chimeric molecules. Recombinant AAV (A) expression of CA89, CA65, VEGF-CΔNΔC and VEGF-C were analysed by immunoprecipitation of metabolically labeled proteins with anti-VEGF-C serum followed by SDS-PAGE under reducing conditions.

FIG. 6B. Analysis of viral expression of the chimeric molecules. Recombinant adenoviral expression of CA89, CA65, VEGF-CΔNΔC and VEGF-C were analysed by immunoprecipitation of metabolically labeled proteins with anti-VEGF-C serum followed by SDS-PAGE under reducing conditions.

### DETAILED DESCRIPTION OF THE INVENTION

1. Vascular Endothelial Growth Factors

Human, non-human mammalian, and avian Vascular Endothelial Growth Factor C (VEGF-C) polynucleotides and polypeptides, as well as VEGF-C variants and analogs, have been described in detail in International Patent Application Number PCT/US98/01973, filed 2 Feb. 1998 and published on 6 Aug. 1998 as International Publication Number WO 98/33917; in PCT Patent Application PCT/FI96/00427, filed Aug. 1, 1996, and published as International Publication WO 97/05250; in related U.S. Pat. Nos. 5,776,755, 6,130,071, 6,221,839, 6,245,530, and 6,361,946; in Joukov et al., J. Biol. Chem., 273(12):6599-6602 (1998); and in Joukov et al., EMBO J., 16(13):3898-3911 (1997), all of which are incorporated herein by reference in their entirety. As explained therein in detail, human VEGF-C is initially produced in human cells as a prepro-VEGF-C polypeptide of 419 amino acids. A cDNA and deduced amino acid sequence for human prepro-VEGF-C are set forth in SEQ ID NOs: 1 and 2, respectively, and a cDNA encoding human VEGF-C has been deposited with the American Type Culture Collection (ATCC), 10801 University Blvd., Manassas, Va. 20110-2209 (USA), pursuant to the provisions of the Budapest Treaty (Deposit date of 24 Jul. 1995 and ATCC Accession Number 97231). VEGF-C sequences from other species have also been reported. See Genbank Accession Nos. MMU73620 (Mus musculus); and CCY15837 (Coturnix coturnix) for example, incorporated herein by reference.

The prepro-VEGF-C polypeptide is processed in multiple stages to produce a mature and most active VEGF-C polypeptide of about 21-23 kD (as assessed by SDS-PAGE under reducing conditions). Such processing includes cleavage of a signal peptide (SEQ ID NO: 2, residues 1-31); cleavage of a carboxyl-terminal peptide (corresponding approximately to amino acids 228-419 of SEQ ID NO: 2; and having a pattern of spaced cysteine residues reminiscent of a Balbiani ring 3 protein (BR3P) sequence (Dignam et al., Gene, 88:133-40 (1990); Paulsson et al., J. Mol. Biol., 211:331-49 (1990)) to produce a partially-processed form of about 29 kD; and cleavage (apparently extracellularly) of an amino-terminal peptide (corresponding approximately to amino acids 32-102 of SEQ ID NO: 2) to produced a fully-processed mature form of about 21-23 kD. A “recombinantly matured” VEGF-C polypeptide comprises amino acids 1-31 of SEQ ID NO: 2 fused in frame with amino acids 103-227 of SEQ ID NO: 2 is shown in SEQ ID NO: 8. The corresponding DNA sequence to the recombinantly matured VEGF-C is shown in SEQ ID NO: 7. Alternatively, a signal sequence other than the native VEGF-C signal sequence (amino acids 1-31 of SEQ ID NO: 2) may be used. Experimental evidence demonstrates that partially-processed forms of VEGF-C (e.g., the 29 kD form) and fully processed forms are able to bind the Flt4 (VEGFR-3) receptor, whereas only fully processed forms of VEGF-C exhibit high affinity binding to VEGFR-2. VEGF-C polypeptides naturally associate as (apparently) non-disulfide linked dimers.

Moreover, it has been demonstrated that amino acids 103-227 of SEQ ID NO: 2 are not all critical for maintaining VEGF-C functions. A polypeptide consisting of amino acids 113-213 (and lacking residues 103-112 and 214-227) of SEQ ID NO: 2 retains the ability to bind and stimulate VEGF-C receptors, and it is expected that a polypeptide spanning from about residue 131 to about residue 211 will retain VEGF-C biological activity. The cysteine at position 165 of SEQ ID NO: 2 is essential for binding either receptor, whereas analogs lacking the cysteines at positions 83 or 137 compete with native VEGF-C for binding with both receptors and stimulate both receptors.

The cysteine residue at position 156 has been shown to be important for VEGFR-2 binding ability. However, VEGF-C156X polypeptides (i.e., analogs that lack this cysteine due to substitution) remain potent activators of VEGFR-3 and are useful for practice of the present invention.

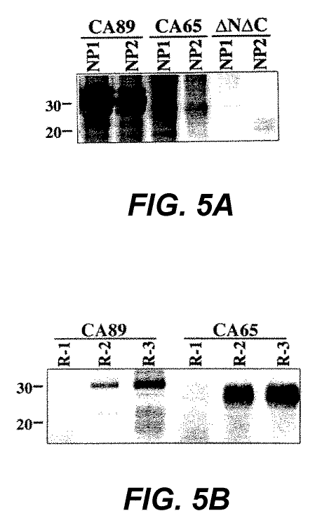

An alignment of human VEGF-C with VEGF-C from other species (performed using any generally accepted alignment algorithm) suggests additional residues wherein modifications can be introduced (e.g., insertions, substitutions, and/or deletions) without destroying VEGF-C biological activity. Any position at which aligned VEGF-C polypeptides of two or more species have different amino acids, especially different amino acids with side chains of different chemical character, is a likely position susceptible to modification without concomitant elimination of function. An exemplary alignment of human, murine, and quail VEGF-C is set forth in FIG. 5 of PCT/US98/01973.

Apart from the foregoing considerations, it will be understood that innumerable conservative amino acid substitutions can be performed to a wildtype VEGF-C sequence which are likely to result in a polypeptide that retains VEGF-C biological activities, especially if the number of such substitutions is small. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine). Addition or deletion of one or a few internal amino acids without destroying VEGF-C biological activities also is contemplated.

Candidate VEGF-C analog polypeptides can be rapidly screened first for their ability to bind and stimulate autophosphorylation of known VEGF-C receptors (VEGFR-2 and VEGFR-3). Polypeptides that stimulate one or both known receptors are rapidly re-screened in vitro for their mitogenic and/or chemotactic activity against cultured capillary or arterial endothelial cells (e.g., as described in WO 98/33917). Polypeptides with mitogenic and/or chemotactic activity are then screened in vivo as described herein for efficacy in methods of the invention. In this way, variants (analogs) of naturally occurring VEGF-C proteins are rapidly screened to determine whether or not the variants have the requisite biological activity to constitute “VEGF-C polypeptides” for use in the present invention.

The growth factor named Vascular Endothelial Growth Factor D (VEGF-D), as well as human sequences encoding VEGF-D, and VEGF-D variants and analogs, have been described in detail in International Patent Application Number PCT/US97/14696, filed 21 Aug. 1997 and published on 26 Feb. 1998 as International Publication Number WO 98/07832; in U.S. Pat. No. 6,235,713; and in Achen, et al., Proc. Nat'l Acad. Sci. U.S.A., 95(2):548-553 (1998), all of which are incorporated herein by reference in the entirety. As explained therein in detail, human VEGF-D is initially produced in human cells as a prepro-VEGF-D polypeptide of 354 amino acids. A cDNA and deduced amino acid sequence for human prepro-VEGF-D are set forth in SEQ ID NOs: 3 and 4, respectively. VEGF-D sequences from other species also have been reported. See Genbank Accession Nos. D89628 (Mus musculus); and AF014827 (Rattus norvegicus), for example, incorporated herein by reference.

The prepro-VEGF-D polypeptide has a putative signal peptide of 21 amino acids and is apparently proteolytically processed in a manner analogous to the processing of prepro-VEGF-C. A “recombinantly matured” VEGF-D polypeptide comprises amino acids 1-25 of SEQ ID NO: 4 fused in frame with amino acids 93-201 of SEQ ID NO: 4 is shown in SEQ ID NO: 10. The corresponding DNA sequence to the recombinantly matured VEGF-C is shown in SEQ ID NO: 9. Alternatively, a signal sequence other than the native VEGF-D signal sequence (amino acids 1-25 of SEQ ID NO: 4) may be used. A recombinantly matured VEGF-D lacking residues 1-92 and 202-354 of SEQ ID NO: 4 retains the ability to activate receptors VEGFR-2 and VEGFR-3, and appears to associate as non-covalently linked dimers. Thus, preferred VEGF-D polynucleotides include those polynucleotides that comprise a nucleotide sequence encoding amino acids 93-201 of SEQ ID NO: 4.

2. Reconstructive and Cosmetic Surgery

Reconstructive surgery is generally performed on abnormal structures of the body, caused by birth defects, developmental abnormalities, trauma or injury, infection, tumors, or disease. It is generally performed to improve function, but may also be done to approximate a normal appearance. Cosmetic surgery is performed to reshape normal structures of the body to improve the patient's appearance and self-esteem.

Complications resulting from reconstructive and cosmetic surgery may include infection; excessive bleeding, such as hematomas (pooling of blood beneath the skin); significant bruising and wound-healing difficulties; pain; edema; and problems related to anesthesia and surgery. The methods and compositions described herein provide a much-needed treatment to improve post-surgical wound healing.

Many common reconstructive and cosmetic surgery procedures result in painful swelling and bleeding where skin flaps and/or grafts are used. In breast augmentation, breast reduction, mastopexy and gynecomastia procedures, for example, fluid accumulation and swelling may result, possibly requiring subsequent corrective surgical procedures. In such procedures, skin of and around the nipple is separated and/or removed from the underlying breast tissue. A skin flap or skin graft is frequently necessary to compensate for the change in breast size and/or to gain access to underlying tissues for implantation or reduction. Accordingly, the methods and compositions of the present invention can be used to promote wound healing prior to, during, and/or following the aforementioned surgical procedures.

Similarly, cosmetic surgery procedures such as rhytidectomy, browlift, otoplasty, blepharoplasty, rhinoplasty, facial implant, and hair replacement therapy will also benefit from the present invention. In such procedures, skin is lifted and underlying tissue and muscles are removed or manipulated. A skin flap or skin graft is frequently necessary to compensate for skin tissue loss and/or to gain access to the tissues and muscles beneath the skin. Accordingly, the methods and compositions of the present invention can be used to promote wound healing prior to, during, and/or following the aforementioned surgical procedures.

In an abdominoplasty procedure, the abdomen is flattened by removing excess fat and skin and tightening muscles of the abdominal wall. Bleeding under the skin flap and poor healing resulting in skin loss and scarring may occur, possibly requiring a second operation. Accordingly, the methods and compositions of the present invention can be used to promote wound healing prior to, during, and/or following the aforementioned surgical procedure.

Reconstructive surgery procedures such as those to repair a birthmark, cleft palate, cleft lip, syndactyly, urogenital and anorectal malformations, craniofacial birth defects, ear and nasal deformitites or vaginal agenesis similarly involve incisions and manipulations in skin and underlying tissues for the restoration of body features. A skin flap or skin graft is frequently necessary to compensate for skin tissue loss and/or to gain access to the tissues and muscles beneath the skin. Accordingly, the methods and compositions of the present invention can be used to promote wound healing prior to, during, and/or following the aforementioned surgical procedures.

Similarly, reconstructive surgery to correct defects resulting from an injury such as a burn, infection, or disease such as skin cancer will also benefit from the compositions and methods of the present invention. For example, an oseomyocutaneous flap (a flap containing bone and soft tissue) is often used to reconstruct the skin following skin cancer excision. Thus, the present invention may be employed to reduce the swelling and scarring complications associated with such a procedure.

3. Skin Flaps and Skin Grafts

A flap is a section of living tissue that carries its own blood supply and is moved from one area of the body to another. Flap surgery can restore form and function to areas of the body that have lost skin, fat, muscle movement, and/or skeletal support.

A local flap uses a piece of skin and underlying tissue that lie adjacent to the wound. The flap remains attached at one end so that it continues to be nourished by its original blood supply, and is repositioned over the wounded area. A regional flap uses a section of tissue that is attached by a specific blood vessel. When the flap is lifted, it needs only a very narrow attachment to the original site to receive its nourishing blood supply from the tethered artery and vein. A musculocutaneous flap, also called a muscle and skin flap, is used when the area to be covered needs more bulk and a more robust blood supply. Musculocutaneous flaps are often used in breast reconstruction to rebuild a breast after mastectomy. This type of flap remains “tethered” to its original blood supply. In a bone/soft tissue flap, bone, along with the overlying skin, is transferred to the wounded area, carrying its own blood supply.

Typically, a wound that is wide and difficult or impossible to close directly may be treated with a skin graft. A skin graft is basically a patch of healthy skin that is taken from one area of the body, called the “donor site,” and used to cover another area where skin is missing or damaged. There are three basic types of skin grafts.

A split-thickness skin graft, commonly used to treat burn wounds, uses only the layers of skin closest to the surface. A full-thickness skin graft might be used to treat a burn wound that is deep and large, or to cover jointed areas where maximum skin elasticity and movement are needed. As its name implies, a full-thickness (all layers) section of skin from the donor site are lifted. A composite graft is used when the wound to be covered needs more underlying support, as with skin cancer on the nose. A composite graft requires lifting all the layers of skin, fat, and sometimes the underlying cartilage from the donor site.

4. Gene Therapy Methods

Delivery of a therapeutic composition of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp. 25-20 (1998). For additional reviews of gene therapy technology see Friedmann, Science, 244:1275-1281 (1989); Verma, Scientific American: 68-84 (1990); and Miller, Nature, 357: 455-460 (1992). Introduction of any one of the polynucleotides of the present invention or a gene encoding the polypeptides of the present invention can also be accomplished with extrachromosomal substrates (transient expression) or artificial chromosomes (stable expression). Transient expression is preferred. Cells may also be cultured ex vivo in the presence of therapeutic compositions of the present invention in order to proliferate or to produce a desired effect on or activity in such cells. Treated cells can then be introduced in vivo for therapeutic purposes.

5. Routes and Administration

The therapeutic compositions are administered by any route that delivers an effective dosage to the desired site of action, with acceptable (preferably minimal) side-effects. Numerous routes of administration of agents are known, for example, oral, rectal, transmucosal, or intestinal administration; parenteral delivery, including intramuscular, subcutaneous, intramedullary injections, as well as intrathecal, intraperitoneal, intranasal, cutaneous or intradermal injections; inhalation, and topical application. However, localized routes or administration directed to the skin and its blood and lymphatic vasculature are preferred. Thus, intradermal administration to the subject is preferred.

Therapeutic dosing is achieved by monitoring therapeutic benefit in terms of any of the parameters outlined herein (speed of wound healing, reduced edema, reduced complications, etc.) and monitoring to avoid side-effects. Preferred dosage provides a maximum localized therapeutic benefit with minimum local or systemic side-effects. Side effects to monitor include blood or lymphatic vessel growth and/or fluid build-up in areas outside those being treated, including the heart. Suitable human dosage ranges for the polynucleotides or polypeptides can be extrapolated from these dosages or from similar studies in appropriate animal models. Dosages can then be adjusted as necessary by the clinician to provide maximal therapeutic benefit for human subjects.

The dosage regimen of a protein-containing composition to be used in tissue regeneration will be determined by the attending physician considering various factors which modify the action of the proteins, e.g., amount of tissue weight desired to be formed, the location of the tissue, the condition of the tissue, the size of the tissue area (e.g., size of a wound), type of tissue (e.g., bone), the patient's age, sex, and diet, the severity of any infection, time of administration and other clinical factors. The dosage may vary with the type of matrix used in the reconstitution and with inclusion of other proteins in the composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations, fluorescence microscopy, and tetracycline labeling.

6. Compositions and Formulations

Compositions for use in accordance with the present invention may be formulated in a conventional manner using one or more physiologically acceptable carriers comprising excipients and auxiliaries which facilitate processing of a therapeutic composition into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

When a therapeutically effective amount of a composition of the present invention is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art. A preferred composition should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The composition of the present invention may also contain stabilizers, preservatives, buffers, antioxidants, or other additives known to those of skill in the art. The agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compounds with pharmaceutically acceptable carriers well known in the art. Such carriers enable the compounds of the invention to be formulated as tablets, pills, dragees, powders, capsules, liquids, solutions, gels, syrups, slurries, suspensions and the like, for oral ingestion by a patient to be treated.

For administration by inhalation, the compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas.

Pharmaceutical formulations for parenteral administration include aqueous solutions of the compositions in water-soluble form. Optionally, the suspension may also contain suitable stabilizers or agents which increase the solubility of the compositions to allow for the preparation of highly concentrated solutions. Alternatively, the active ingredient may be in powder form for constitution with a suitable vehicle, e.g., sterile pyrogen-free water, before use.

Polypeptides and/or polynucleotides of the invention may be administered in any suitable manner using an appropriate pharmaceutically acceptable vehicle, e.g., a pharmaceutically acceptable diluent, adjuvant, excipient or carrier. The composition to be administered according to methods of the invention preferably comprises (in addition to the polynucleotide or vector) a pharmaceutically acceptable carrier solution such as water, saline, phosphate buffered saline, glucose, or other carriers conventionally used to deliver therapeutics intravascularly. Multi gene therapy is also contemplated, in which case the composition optionally comprises both the polynucleotide of the invention/vector and another polynucleotide/vector selected to prevent restenosis or other disorder mediated through the action of a VEGF receptor. Exemplary candidate genes/vectors for co transfection with transgenes encoding polypeptides of the invention are described in the literature cited above, including genes encoding cytotoxic factors, cytostatic factors, endothelial growth factors, and smooth muscle cell growth/migration inhibitors.

The “administering” that is performed according to the present method may be performed using any medically-accepted means for introducing a therapeutic directly or indirectly into the vasculature of a mammalian subject, including but not limited to injections (e.g., intravenous, intramuscular, subcutaneous, or catheter); oral ingestion; intranasal or topical administration; and the like. In a preferred embodiment, administration of the composition comprising a polynucleotide of the invention is performed intravascularly, such as by intravenous, intra-arterial, or intracoronary arterial injection. The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly. To minimize angiogenic side effects in non-target tissues, preferred methods of administration are methods of local administration, such as admistration by intramuscular injection.

In general, peroral dosage forms for the therapeutic delivery of polypeptides is ineffective because in order for such a formulation to the efficacious, the peptide must be protected from the enzymatic environment of the gastrointestinal tract. Additionally, the polypeptide must be formulated such that it is readily absorbed by the epithelial cell barrier in sufficient concentrations to effect a therapeutic outcome. The chimeric polypeptides of the present invention may be formulated with uptake or absorption enhancers to increase their efficacy. Such enhancer include for example, salicylate, glycocholate/linoleate, glycholate, aprotinin, bacitracin, SDS caprate and the like. An additional detailed discussion of oral formulations of peptides for therapeutic delivery is found in Fix, J. Pharm. Sci., 85(12) 1282 1285, 1996, and Oliyai and Stella, Ann. Rev. Pharmacol. Toxicol., 32:521 544, 1993, both incorporated by reference.

The amounts of peptides in a given dosage will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day. These concentrations may be administered as a single dosage form or as multiple doses.

The polypeptides may also be employed in accordance with the present invention by expression of such polypeptide in vivo, which is often referred to as gene therapy. The present invention provides a recombinant DNA vector containing a heterologous segment encoding a chimeric polypeptide of the invention that is capable of being inserted into a microorganism or eukaryotic cell and that is capable of expressing the encoded chimeric protein.

In still another variation, endothelial cells or endothelial progenitor cells are transfected ex vivo with the transgene encoding a polypeptide of the invention, and the transfected cells as administered to the mammalian subject. Exemplary procedures for seeding a vascular graft with genetically modified endothelial cells are described in U.S. Pat. No. 5,785,965, incorporated herein by reference.

In preferred embodiments, polynucleotides of the invention further comprises additional sequences to facilitate the gene therapy. In one embodiment, a “naked” transgene encoding a polypeptide of the invention (i.e., a transgene without a viral, liposomal, or other vector to facilitate transfection) is employed for gene therapy. In this embodiment, the polynucleotide of the invention preferably comprises a suitable promoter and/or enhancer sequence (e.g., cytomegalovirus promoter/enhancer [Lehner et al., J. Clin. Microbiol., 29:2494 2502 (1991); Boshart et al., Cell, 41:521 530 (1985)]; Rous sarcoma virus promoter [Davis et al., Hum. Gene Ther., 4:151 (1993)]; Tie promoter [Korhonen et al., Blood, 86(5): 1828 1835 (1995)]; or simian virus 40 promoter) for expression in the target mammalian cells, the promoter being operatively linked upstream (i.e., 5′) of the polypeptide coding sequence. The polynucleotides of the invention also preferably further includes a suitable polyadenylation sequence (e.g., the SV40 or human growth hormone gene polyadenylation sequence) operably linked downstream (i.e., 3′) of the polypeptide coding sequence. The polynucleotides of the invention also preferably comprise a nucleotide sequence encoding a secretory signal peptide fused in frame with the polypeptide sequence. The secretory signal peptide directs secretion of the polypeptide of the invention by the cells that express the polynucleotide, and is cleaved by the cell from the secreted polypeptide. The signal peptide sequence can be that of another secreted protein, or can be a completely synthetic signal sequence effective to direct secretion in cells of the mammalian subject.

The polynucleotide may further optionally comprise sequences whose only intended function is to facilitate large scale production of the vector, e.g., in bacteria, such as a bacterial origin of replication and a sequence encoding a selectable marker. However, in a preferred embodiment, such extraneous sequences are at least partially cleaved off prior to administration to humans according to methods of the invention. One can manufacture and administer such polynucleotides for gene therapy using procedures that have been described in the literature for other transgenes. See, e.g., Isner et al., Circulation, 91: 2687-2692 (1995); and Isner et al., Human Gene Therapy, 7: 989-1011 (1996); incorporated herein by reference in the entirety.

Any suitable vector may be used to introduce the transgene encoding one of the polypeptides of the invention, into the host. Exemplary vectors that have been described in the literature include replication deficient retroviral vectors, including but not limited to lentivirus vectors [Kim et al., J. Virol., 72(1): 811-816 (1998); Kingsman & Johnson, Scrip Magazine, October, 1998, pp. 43 46.]; adeno associated viral vectors [U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479; Gnatenko et al., J. Investig. Med., 45: 87 98 (1997)]; adenoviral vectors [See, e.g., U.S. Pat. No. 5,792,453; U.S. Pat. No. 5,824,544; U.S. Pat. No. 5,707,618; U.S. Pat. No. 5,693,509; U.S. Pat. No. 5,670,488; U.S. Pat. No. 5,585,362; Quantin et al., Proc. Natl. Acad. Sci. USA, 89: 2581 2584 (1992); Stratford Perricadet et al., J. Clin. Invest., 90: 626 630 (1992); and Rosenfeld et al., Cell, 68: 143 155 (1992)]; an adenoviral adenoassociated viral chimeric (see for example, U.S. Pat. No. 5,856,152) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688; Lipofectin mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in their entirety. Replication deficient adenoviral vectors constitute a preferred embodiment.

Other non-viral delivery mechanisms contemplated include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am. 276(6):102 6, 1997; Felgner, Hum Gene Ther. 7(15):1791 3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

The expression construct (or indeed the polypeptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, Wu G, Wu C ed., New York: Marcel Dekker, pp. 87-104, 1991). The addition of DNA to cationic liposomes causes a topological transition from liposomes to optically birefringent liquid-crystalline condensed globules (Radler et al., Science, 275(5301):810 4, 1997). These DNA-lipid complexes are potential non-viral vectors for use in gene therapy and delivery.

Liposome-mediated nucleic acid delivery and expression of foreign DNA in vitro has been successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993, supra).

In other embodiments, the delivery vehicle may comprise a ligand and a liposome. For example, Nicolau et al. (Methods Enzymol., 149:157-176, 1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky et al. (Proc. Nat. Acad. Sci. USA, 81:7529-7533, 1984) successfully injected polyomavirus DNA in the form of CaPO4 precipitates into liver and spleen of adult and newborn mice demonstrating active viral replication and acute infection. Benvenisty and Neshif (Proc. Nat. Acad. Sci. USA, 83:9551-9555, 1986) also demonstrated that direct intraperitoneal injection of CaPO4 precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

In embodiments employing a viral vector, preferred polynucleotides still include a suitable promoter and polyadenylation sequence as described above. Moreover, it will be readily apparent that, in these embodiments, the polynucleotide further includes vector polynucleotide sequences (e.g., adenoviral polynucleotide sequences) operably connected to the sequence encoding a polypeptide of the invention.

Thus, in one embodiment the composition to be administered comprises a vector, wherein the vector comprises a polynucleotide of the invention. In a preferred embodiment, the vector is an adenovirus vector. In a highly preferred embodiment, the adenovirus vector is replication deficient, i.e., it cannot replicate in the mammalian subject due to deletion of essential viral replication sequences from the adenoviral genome. For example, the inventors contemplate a method wherein the vector comprises a replication deficient adenovirus, the adenovirus comprising the polynucleotide of the invention operably connected to a promoter and flanked on either end by adenoviral polynucleotide sequences.

Similarly, the invention includes kits which comprise compounds or compositions of the invention packaged in a manner which facilitates their use to practice methods of the invention. In a simplest embodiment, such a kit includes a compound or composition described herein as useful for practice of the invention (e.g., polynucleotides or polypeptides of the invention), packaged in a container such as a sealed bottle or vessel, with a label affixed to the container or included in the package that describes use of the compound or composition to practice the method of the invention. Preferably, the compound or composition is packaged in a unit dosage form. In another embodiment, a kit of the invention includes a composition of both a polynucleotide or polypeptide packaged together with a physical device useful for implementing methods of the invention, such as a stent, a catheter, an extravascular collar, a polymer film, or the like. In another embodiment, a kit of the invention includes compositions of both a polynucleotide or polypeptide of the invention packaged together with a hydrogel polymer, or microparticle polymers, or other carriers described herein as useful for delivery of the polynucleotides or polypeptides to the patient.

The compositions may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions also may comprise suitable solid or gel phase carriers or excipients.

The compositions of the invention may be in the form of a complex of the protein(s) or other active ingredient of present invention along with protein or peptide antigens.

The compositions may include a matrix capable of delivering the protein-containing or other active ingredient-containing composition to the site of tissue damage, providing a structure for the developing bone and cartilage and optimally capable of being resorbed into the body. Such matrices may be formed of materials presently in use for other implanted medical applications. The choice of matrix material is based on biocompatibility, biodegradability, mechanical properties, cosmetic appearance and interface properties.

In further compositions, proteins or other active ingredient of the invention may be combined with other agents beneficial to the treatment of the bone and/or cartilage defect, wound, or tissue in question.

The composition may further contain other agents which either enhance the activity of the protein or other active ingredient or complement its activity or use in treatment. Such additional factors and/or agents may be included in the pharmaceutical composition to produce a synergistic effect with protein or other active ingredient of the invention, or to minimize side effects. VEGF-C and -D proteins form dimers and as a result, pharmaceutical compositions of the invention may comprise a protein of the invention in such multimeric or in complexed forms.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

7. Transdermal Patch

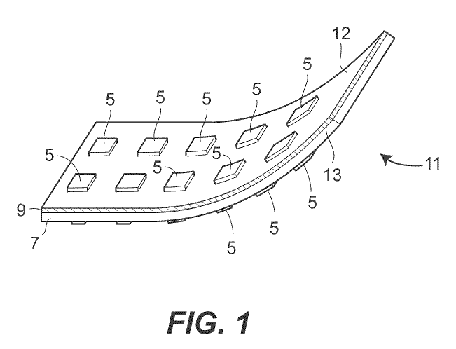

A transdermal patch may be employed to deliver VEGF-C or VEGF-D compositions to practice the invention. FIG. 1 is representative of a suitable patch for the delivery of therapeutic compositions according to some embodiments of the invention. The patch 11 includes a pad 9 having an upper surface area 12 and a lower surface area 13; an adhesive 7 on the lower surface area 13 of the pad 9, and an agent 5 for delivery to the skin of a subject. The patch will include, but is not limited to, a pad material, adhesive, and therapeutic composition. The pad material which is useful for this invention is not particularly limited as long as it can provide a suitable substrate for the adhesive and is sufficiently strong to withstand removal from the skin, having been secured to the skin by adhesive. In some embodiments, the pad should provide a suitable substrate for the formation of apertures therein.

The pad material is preferably flexible from the viewpoint of comfort. The flexibility is achievable by elasticity in any one or all axes of the material. Examples of flexible materials include, but are not limited to cotton cloth, rayon cloth, tetron cloth, nylon cloth or plastic foam. The pad material is preferably pliable to accommodate skin contours, when applied to areas of skin having alterations in surface angles (for example around the nostril skin area). The pad is preferably non-stretchable, namely non-elastic, in the planar axis of the material.

The pad material is also preferably breathable, thereby allowing air to pass through the patch and contact the skin. In some embodiments, however, the pad may not breathable. The pad material is also preferably not permeable to the agent applied to the patch. However, in some embodiments it is preferable that the pad be permeable to the agent. The pad material is also preferably of a thickness to provide sufficient strength to the pad, but also of a thinness which will be comfortable to the wearer and pliable to contact all skin surfaces.

An adhesive useful in this invention is any substance which holds the patch in contact with the skin.

In a preferred embodiment, the agent can be applied to the patch in discrete locations. The therapeutic composition is preferably present in an amount and a concentration such that an effective dose of the agent will be applied to the skin over the designated time that the patch remains adhered to the skin. The dosage of agent available to the skin may be altered by altering the density of the discrete applications of the primary agent to the defined surface area of the patch, the cross-sectional area of each application for a defined surface area of the patch, the cavity volume (as measured by the depth and cross-sectional area) of the aperture containing the agent in a defined surface area of the patch, or any combination of these parameters described. Thus, where a liner is used as a mask in adding agent to the patch, the greater the depth of the apertures in the liner, the greater the amount of agent available for delivery to the skin. Similarly, the greater the density of apertures, or the cross sectional area of the apertures, the greater the amount of agent available for delivery to the skin.

Delivery of the therapeutic composition to the skin may proceed by a process including, but not limited to, liquefaction upon moisturization of the composition, diffusion of the agent away from the patch or capillary action of the composition from the patch to the skin.

The following examples assist in further describing the invention, but are not intended in any way to limit the scope of the invention.

### EXAMPLE 1

### Expression of Virally Transduced Genes In Vitro and In Vivo

The following example describes the synthesis of recombinant viral vectors for expression of VEGF-C and VEGF-C156S and assays to demonstrate that cells transfected with the vector produce the desired proteins.

A. Generation and In Vitro Analysis of Recombinant Adenoviruses and AAVs

The adenovirus construct AdVEGF-C156S was cloned as described in Saaristo et al., J. Exp. Med., 196: 719-730 (2002). Briefly, the human VEGF-C156S cDNA of SEQ ID NO: 5 was cloned as a BamHI/NotI fragment into the corresponding sites of the pAdBglII vector. Replication-deficient E1-E3 deleted adenoviruses were produced in 293 cells and concentrated by ultracentrifugation (Puumalainen, A. M., et al., Hum. Gene Ther., 9:1769-1774 (1998)). Adenoviral preparations were analyzed to be free of helper viruses, lipopolysaccharide, and bacteriological contaminants (Laitinen, M., et al., Hum. Gene Ther., 9:1481-1486 (1998)). The adenoviruses encoding human VEGF-C (AdVEGF-C) and nuclear targeted LacZ (Ad-LacZ) were constructed as described in Enholm, B., et al., Circ. Res., 88:623-629 (2001); and Puumalainen, A. M., et al., supra. Briefly, for Ad-VEGF-C, a full-length human VEGF-C cDNA (GenBank accession No. X94216) (SEQ ID NO: 1) was cloned under the cytomegalovirus promoter in the pcDNA3 vector (Invitrogen). The SV40-derived polyadenylation signal of the vector was then exchanged for that of the human growth hormone gene, and the transcription unit was inserted into the pAdBglII vector as a BamHI fragment. Replication-deficient recombinant E1-E3-deleted adenoviruses were produced in human embryonic kidney 293 cells and concentrated by ultracentrifugation as previously described (Puumalainen, et al., supra). Similarly, recombinant adenovirus encoding VEGF165 was constructed as previously described (Makinen, et al., Mol. Ther., 6(1):127-133 (2002)). Adenoviral preparations were confirmed to be free from helper viruses, lipopolysaccharide, and bacteriological contaminants.

AAV-VEGF-C156S construct was cloned as described in Saaristo et al J. Exp. Med (2002) 196 719-30). Briefly, the full-length human VEGF-C 156S was cloned as a blunt-end fragment into the MluI site of psub-CMV-WPRE plasmid and the rAAV type 2 was produced as described in Karkkainen, M. J., et al., Proc. Natl. Acad. Sci. USA., 98:12677-12682 (2001). Construction of AAV-VEGF-C and a control AAV encoding Enhanced Green Fluorescent Protein (EGFP), AAV-EGFP, is described in Karkkainen, M. J., et al., supra; Paterna, J. C., et al., Gene Ther., 7:1304-1311 (2000).

For the analysis of protein expression, 293EBNA cells were infected with recombinant adenoviruses for 2 hours in serum-free medium or by AAVs for 8 hours in 2% FCS medium. After 24-72 hours, the cells were metabolically labeled for 8 hours and subjected to immunoprecipitation with VEGF-C-specific antibodies or to a binding assay using soluble VEGFR-2-Ig (R&D Systems) and VEGFR-3-Ig (Achen, et al., Proc. Nat'l Acad. Sci. U.S.A., 95(2):548-553 (1998)) fusion proteins. AdLacZ and AAV-EGFP infected cells were used as negative controls. The bound proteins were precipitated with protein G Sepharose, separated in 15% SDS-PAGE, and analyzed by autoradiography. To compare the protein production levels of AdVEGF-C156S and AdVEGF-C viruses, 20-μl aliquots of the media from AdVEGF-C156S, AdVEGF-C, and AdLacZ infected cell cultures were separated in 15% SDSPAGE gel and subjected to Western blotting using polyclonal anti-VEGF-C antibodies (R&D Systems).

B. In Vivo Use and Analysis of the Viral Vectors

5×10⁸ pfu of the recombinant adenoviruses or 5×10⁹-1×10¹¹ rAAV particles were injected intradermally into the ears of NMRI nu/nu mice (Harlan) or Chy lymphedema mice (Karkkainen, M. J., et al., supra). The infected nude mice were killed 3, 5, 7, 10, 14, 21, 42, or 56 days after adenoviral infection and 3, 6, or 8 wk after AAV infection. The AAV-infected Chy mice were killed 1, 2, 4, 6, or 8 months after infection. Total RNA was extracted from the ears (RNAeasy Kit; QIAGEN) 1 to 8 weeks after adenoviral infection and 10 weeks after AAV-infection. 10 μg of RNA was subjected to Northern blotting and hybridization with a mixture of [α32P]dCTP (Amersham Biotech) labeled cDNAs specific for VEGF-C. The glyceraldehyde-3-phosphate dehydrogenase cDNA probe was used as an internal control for equal loading. The adenoviral protein expression was confirmed by whole mount β-galactosidase staining (Prui, M. C., et al., EMBO J., 14:5884-5891 (1995)) of the AdLacZ-infected ears 1 to 7 weeks after gene transfer. The AAV-EGFP-infected ears were studied under the fluorescence microscope at 3 weeks to 8 months after infection.

C. Results

The production of active VEGF-C156S and VEGF-C proteins into the cell culture media of recombinant adenovirus (Ad)-or AAV-infected, metabolically labeled 293EBNA cells was confirmed by immunoprecipitation and by binding to soluble VEGFR-2-Ig and VEGFR-3-Ig fusion proteins. Both the partially processed 30 kD and the fully processed 21-kD forms of VEGF-C156S and VEGF-C were observed, and both forms of VEGF-C156S and VEGF-C bound to VEGFR-3-Ig, but only the 21-kD form of VEGF-C was capable of binding to VEGFR-2-Ig. Furthermore, Western blotting analysis of media from the infected cultures confirmed that the same viral titers of AdVEGF-C156S and AdVEGF-C gave rise to comparable levels of the corresponding proteins in vitro.

To analyze the expression of adenovirus and AAV transduced genes in vivo, RNA samples from infected mouse ear skin were analyzed by Northern blotting. High levels of human VEGF-C156S and VEGF-C mRNAs were detected in the AdVEGF-C156S and AdVEGF-C infected tissues 1 wk after infection. 3 weeks after infection transgene expression in the control AdLacZ infected ears was still strong. Thereafter the transgene expression was gradually down-regulated, and by 8 weeks expression was no longer detected in the adenovirus-infected ear. Somewhat weaker, but more sustained mRNA and protein expression was obtained with the recombinant AAV vectors. Furthermore, at 8 months after infection, the latest time point studied, EGFP fluorescence was still detected in the ear skin of the Chy mice infected with the AAV-EGFP control virus.

### EXAMPLE 2

### Skin Flap Model

The following example describes the use of VEGF-C156S and VEGF-C adenoviral vectors to improve healing and reduce post-surgical complications in a skin flap operation procedure.

A. Operative Technique

NMRI nu/nu mice (Harlan, Horst, Netherland) were anesthetized and an epigastric flap was made to the ventral skin. The epigastric flap was elevated after incising the distal, proximal, and lateral borders. The flap elevation was performed with small scissors and no hemostasis was required. The right inferior epigastric vessels were incised and only the left inferior epigastric vessel remained functional in the flap pedicle. Finally, the flap was sutured back to its native configuration by using interrupted 5-0 non-absorbable sutures.

B. Administration and Evaluation of Adenoviral Vectors

The adenoviruses encoding VEGF-C, VEGF-C156S or LacZ were described in Example 1. 1×10⁹ pfu of adenoviral particles were injected intradermally into the ventral skin to the site of the epigastric flap surgery of NMRI nu/nu mice and the mice were sacrificed 2 weeks after the infection.

C. Follow-Up

Fluorescent FITC-dextran was injected to the flap skin of the mice 2 weeks after AdVEGF-C, AdVEGF-C156S or AdLacZ infection. Functional lymphatic vessels in VEGF-C and VEGF-C156S treated mice were observed, while lymphatic vessels were virtually absent in the LacZ control. After FITC-dextran injection, axillary lymphnodes ipsilateral to the side of dextran injection were revealed and accumulation of dextran visualized under a fluorescent microscope. Accumulation of fluorescent dextran in the lymph node was observed only in mice treated with adenoviral VEGF-C and VEGF-C156S, indicating that the VEGF-C and VEGF-C156S had caused an increase in functional lymphatic vessels. Tissue sections harvested from the flap margin were stained against VEGFR-3 in order to visualize lymphatic vessels in the area of the incision. Prominent lymphatic vasculature in VEGF-C and VEGF-C156S treated flaps, even at the site of the incision, were observed, while a corresponding sample from the control group contained very few lymphatic vessels.

Additional analysis of the animals in this type of study is contemplated and would provide further useful data. For example, follow-up evaluation is performed on postoperative days 7 and 14. The animals are anesthetized and placed prone on a scanner bed. Digital images of epigastric flaps are scanned to the computer. The following flap zones are defined for surface area measurement: necrotic zone (representing apparently nonviable eschar), hypoxic zone (excoriated skin with hair loss), and total flap area (defined by the surgical borders). Surface area of these defined zones is measured by using, for example, Image Pro Plus Software (version 4.1, Media Cybernetics LP, Silver Spring, Md.). The results are expressed as percentages relative to total flap surface area. Animals are sacrificed after evaluation of adenoviral VEGF-C156S expression with an overdose of intraperitoneal pentobarbital (100 mg/kg) and skin specimens are taken and stained with hematoxylin and eosin for histologic evaluation.

D. Summary

The aforementioned model demonstrates the therapeutic potential of using VEGF-C and VEGF-C156S to preserve function of the lymphatic vessels and to improve healing and reduce edema and concomitant post-surgical complications in the skin flaps. Thus, the procedures and compositions described herein provide an important need in the art. Specifically, the reduction of edema or increase in perfusion at a skin graft or skin flap can be accomplished, for example, by delivery of AdVEGF-C or AdVEGF-C156S to the site of the surgery.

### EXAMPLE 3

### VEGF-C Gene Therapy Restores Lymphatic Flow Across Incision Wound

A. Administration and Evaluation of Adenoviral Vectors

This example, similar to Example 2, shows that vascular endothelial growth factor-C (VEGF-C) gene transfer can be used to reconstruct a lymphatic vessel network severed by incision of skin flaps. Adenoviral VEGF-C gene transfer was employed at the edges of the epigastric skin flaps in mice.

Adenoviruses encoding human VEGF-C, VEGF-C156S and LacZ were constructed and protein expression tested as described in Example 1. NMRI nu/nu mice were anesthetized with intraperitoneal injection of xylazine (10 mg/kg)and ketamine (50 mg/kg). For analgesia, mice received buprenorphine 0.1-0.5 mg/kg subcutaneously twice per day. The vascular pedicle of the epigastric flap employed the right inferior epigastric artery and vein. When the whole flap was elevated, adenoviral vectors encoding either VEGF-C, VEGF-C156S or LacZ control virus (5×10⁸ pfu) were injected intradermally into the whole distal edge of the flap. Finally the flap was sutured back to the original position.

The flaps were analyzed at 2 weeks, 1 month or 2 months after the operation. At least five mice were used in each study group for each analytical technique and time point. To study the function of the cutaneous lymphatic vessels, a small volume of FITC-labeled dextran (MW 2,000,000; Sigma) was injected intradermally into the cranial edge of the skin flap. Drainage of the dye via the lymphatic vessels into the axillary lymph nodes was followed under a fluorescence microscope.

After microlymphangiography analysis, the mice were sacrificed and four standard skin samples were dissected from the wound area in the cranial margin of the flap. RNA isolation and Northern analysis of VEGF-C mRNA expression was carried out as described in Example 1. In addition, the tissues were fixed and deparaffinized sections were immunostained for VEGFR-3 and for the pan-endothelial marker, PECAM-1 (BD Pharmingen). To quantify the number of lymphatic and blood vessels in the flap wound, at least three vessel hot spot areas (4 mm² diameter) were chosen from five different samples in each study group (with different virus and time point). Only the healing wound areas were used for this analysis. VEGFR-3 or PECAM-1 positive vessels were then counted under a microscope.

B. Results

High levels of human VEGF-C and VEGF-C156S mRNAs were detected by Northern analysis in the AdVEGF-C and AdVEGF-C156S injected flaps two weeks after gene transfer, despite the fact that the adenoviral gene expression is strongest one week after gene transduction, after which the expression levels gradually decline within a month. The mice were analyzed two weeks to two months after the operation. Necrotic or inflamed tissue was not detected in the flaps in any study group. When FITC-dextran was injected into the cranial edge of the flap that had been transduced with adenoviral VEGF-C or VEGF-C156S, a network of FITC-positive lymphatic vessels was detected and some of these vessels drained across the incision wound. In the AdLacZ infected control samples, only few functional lymphatic vessels were present. Two weeks after the operation, FITC-dextran drainage into the axillary lymph nodes was detected in 75-80% of the VEGF-C or VEGF-C156S treated mice and at later time points, in 100% of the mice. In contrast, the corresponding figures were 12.5% and 20-33% in the AdLacZ control group.

Immunohistochemical analysis of the flap margins demonstrated numerous large VEGFR-3 positive lymphatic vessels near the incision area in the AdVEGF-C or AdVEGF-C156S treated mice. In contrast, only few small lymphatic vessels were observed around the incision area in the AdLacZ infected mice. When adjacent tissue sections were stained for the blood vessel marker, PECAM-1, no significant differences were evident between the different study groups.

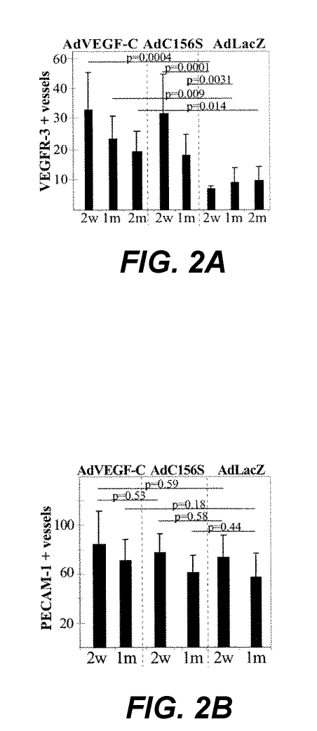

Quantification of the lymphangiogenic and angiogenic responses by counting the number of VEGFR-3 or PECAM-1 positive vessels indicated that AdVEGF-C and AdVEGF-C156S induced a significant increase in the number of lymphatic vessels in comparison to the AdLacZ control (FIG. 2), but a number of the lymphatic vessels regressed after the cessation of adenoviral expression. In contrast, in the control samples, the number of VEGFR-3 positive lymphatic vessels slowly increased during the follow-up period (FIG. 2). However, even when analyzed at the 2 month time point, the lymphatic vessels in the incision area were 1.8-fold more numerous in the AdVEGF-C treated mice than in the AdLacZ treated controls. Quantification of the PECAM-1 positive vessels indicated a small increase in the number of blood vessels in the AdVEGF-C treated flaps when compared to the VEGF-C156S or AdLacZ treated flaps, but these differences were not statistically significant.

B. Summary

This Example demonstrates that pro-lymphangiogenic VEGF-C or VEGF-C156S gene therapy can be used to reconstruct the lymphatic vessel network severed by an incision wound in free flap operations. As shown herein, VEGF-C gene expression results in the formation of anastomoses between the lymphatic vessels of the skin flap and the surrounding lymphatic vasculature. Some spontaneous lymphangiogenesis also took place in the control mice but the lymphatic vessels generated remained nonfunctional even two months post operation. In contrast, the VEGF-C treated mice demonstrated persistent lymphatic vessel function during the two-month follow-up despite the transient nature of the adenoviral VEGF-C gene expression. The restoration of lymphatic function by VEGF-C in skin flaps provides new tools to promote vascular perfusion and to reduce tissue edema in skin and muscle flaps. These results have important implications for the prevention and treatment of surgically induced secondary lymphedema.

### EXAMPLE 4

### Ex Vivo VEGF-C or VEGF-D Gene Transfer to Increase Lymphatic Drainage

This example shows that ex vivo VEGF-C or VEGF-D gene transfer can be used in therapeutic applications to increase lymphatic drainage, e.g., in secondary lymphedema. Secondary lymphedema commonly occurs in patient when the axillary lymph nodes are removed in breast cancer operation.

A. Adenoviral Ex Vivo Transfection of Mouse Embryonic Fibroblasts

Mouse embryonic fibroblasts (MEFs) extracted from ICR mouse embryos were cultured in Dulbecco's Modified Eagle's Medium (DMEM) supplemented with 10% fetal calf serum (FCS), penicillin/streptomycin, and L-glutamine. The MEFs (5^{th} passage; 2.9×10⁶ cells on Ø15 cm plates) were infected with adenoviruses encoding β-galctosidase (AdLacZ), hVEGF165 (AdVEGF), full-length (FL) hVEGF-C (AdVEGF-C), or a recombinantly processed form (ΔN ΔC) of hVEGF-D (AdVEGF-D) as described in Puumalainen, A. M. et al., Hum. Gene Ther. 9, 1769-1774 (1998); Laitinen, M., et al., Hum. Gene Ther. 8, 1737-1744 (1997); Enholm, B., et al., Circ. Res. 88, 623-629 (2001); and Rissanen, T. T., et al., Circ. Res. 92, 1098-1106 (2003).

For viral infection, the cells were first washed with PBS and serum-free DMEM containing 0.2% bovine serum albumin (BSA). The cells were infected with adenoviruses (750 PFU/cell) in 6 ml of serum-free DMEM (0.2%BSA) for two hours at 37° C. The cells were then washed three times with PBS and cultured in normal medium. At 24 hours after infection, the cells were trypsinized and subjected to Matrigel™ implantation. Small aliquots of the cells (about 1.5×10⁵ cells) were plated on 6-well plates for further in vitro analysis of the protein expression. For Matrigel™ implantation, the cells (approximately 3×10⁶ cells/plate) were suspended into 50 μl PBS and 200 μl of Matrigel™ was added to the suspension (on ice). Approximately 1.5×10⁶ cells in the Matrigel™ suspension was implanted to each axilla upon removal of the lymph nodes.

B. In Vitro Characterization of the Protein Production by the Adenovirally Infected MEFs

At the time of Matrigel™ implantation, aliquots of the adenovirally-infected MEFs were subcultured on 6-well plates and analyzed for their protein expression. The transgene expression was analyzed by β-galacatosidase staining (for LacZ expression) or by metabolic labeling and immunoprecipitation (for VEGFs). For β-galacatosidase staining, the cells were washed with PBS and fixed with 0.05% glutaraldehyde in PBS for 15 min at room temperature. The cells were then washed with PBS and incubated with X-gal staining solution [1 mg/ml X-gal (5-bromo-4-chloro-3-indolyl-β-D-galactopyranoside; Sigma) in a solution containing 5 mM K-hexacyanoferrat (II), 5 mM K-hexacyanoferrat (III), 2 mM MgCl2, 0.01% deoxycholic acid sodium salt, 0.02% Nonidet P-40, and 0.1 M phosphate buffer, pH 7.3] for 1 hour at 37° C. The cells were washed with PBS and fixed overnight with 4% PFA in PBS at 4° C. and stained with Nuclear red solution. To analyze the expression of virally produced VEGF proteins, the adenovirus-infected MEFs were metabolically labeled. The cells were first washed with Methionine/Cysteine-free medium and subsequently incubated with 100 μCi/ml [³⁵S ]Met/[³⁵S ]Cys (Promix, Amerham) for 15 hours. The medium was then collected and immunoprecipitated with antibodies against VEGF (cat. MAB293NA), VEGF-C (cat. AF752) or VEGF-D (cat. MAB286) (all antibodies were from R&D Systems) and protein A sepharose (PAS). The PAS beads were washed three times with PBS/0.5% Tween-20 and subjected to 12.5% SDS-PAGE analysis. The gel was dried and exposed on X-ray film.

C. Axillary Lymph Node Removal in Mice

A mouse model was generated mimicking complete lymph node dissection. 6-weeks old female NMRI nu/nu mice were anesthesized with intraperitoneal injection of xylazine (10 mg/kg) and ketamine (50 mg/kg). In order to visualize the axillary lymph nodes, 3% Evans blue dye was injected intradermally into the fore paws of the mice. After 15 min, the axillary lymph nodes were removed. 100 μl of Matrigel™ (BD Biosciences) containing adenovirally transfected MEFs were implanted to the axilla and the axilla was sutured. For post-operative analgesia, the mice received buprenorphine 0.1-0.5 mg/kg s.c. daily. Alternatively, 50 μl of the Matrigel™/MEF suspension was injected intradermally into the ear skin of the mice.

D. Analysis of Lymphatic Vessel Function

The lymphatic drainage of the axillary lymphatic vessels was analyzed 10 days after the surgical procedure. The mice were anesthetized as described above. A small volume of FITC-labelled dextran (MW 2 000 000; Sigma) was injected intradermally to the fore paws of the mice. Drainage of the dye via the lymphatic vessels was followed under a fluorescence microscope.

Axillary lymph nodes were removed from mice and adenovirally transfected mouse embryonic fibroblasts (MEFs) were implanted in Matrigel™ matrix, which supports the growth of the transplanted cells. Alternatively, the MEF/Matrigel™ suspension was injected intradermally to the ears of the mice.

The functional and histological analysis of the lymphatic vessels at the sites of cell transplantation was performed 10 days after the implantation. In the functional analysis of the lymphatic drainage, fluorescent (FITC) dextran was injected intradermally to the fore paws of the mice and accumulation of the dye in the collecting lymphatic vessels was visualized in the axillary region. Lymphatic drainage was detected in one of the two (½) VEGF-C treated axillas and in ¾ of the AdVEGF-D treated axillas, but not in AdVEGF (n=2) or AdLacZ (n=2) treated axillas. This result suggests that the expression of the lymphangiogenic growth factors at the site of lymph node removal is able to induce growth of new, functional lymphatic vessels, which make connections to the pre-existing lymphatic vessels.

Histological analysis of the blood and lymphatic vasculature in the axillary region also indicated that the growth factors secreted by MEFs were able to induce growth of new vessels. VEGF-C induced mainly lymphangiogenesis, whereas the short form of VEGF-D (ΔN ΔC) induced both lymphangiogenesis and also angiogenesis. VEGF165 induced only angiogenesis in this model.

E. Immunohistochemical Stainings

The axillary tissues were fixed embedded in paraffin. Deparaffinized, 5 μm sections were immunostained for LYVE-1 (rabbit antiserum)or for the pan-endothelial marker PECAM-1 (BD Pharmingen). The ears were stained by whole mount immunofluorescent staining with LYVE-1 and PECAM-1 antibodies.

In the ear, VEGF-C and VEGF-D induced strong lymphangiogenic response, whereas VEGF165 induced angiogenesis.

F. Summary

This Example shows that ex vivo VEGF-C or VEGF-D gene transfer can be used in therapeutic applications to increase lymphatic drainage, e.g., in secondary lymphedema. Axillary lymph nodes were removed from mice and growth factor producing cells in Matrigel™ matrix were implanted at the site of lymph node removal. As shown herein, VEGF-C and VEGF-D expression results in the formation of new lymphatic vessels in the vicinity of the cells expressing these therapeutic proteins. Thus, this form of pro-lymphangiogenic therapy could be applicable to various conditions in which tissue edema has to be decreased, such as in tissue swelling resulting from reconstructive surgery.

### EXAMPLE 5

### Using VEGF-C Therapy in Reconstructive Surgery Following a Severe Burn or Other Skin Trauma

The following example describes a procedure and delivery of VEGF-C156S and VEGF-C adenoviral vectors to tissue traumatized from a burn to improve healing following reconstructive surgery. Burn victims often require extensive surgical interventions that include substantial skin grafts to restore damaged tissue. The following example provides a method to improve tissue healing following reconstructive surgery for a burn or other skin trauma.

A. Animals and Skin Preparation

New Zealand white rabbits have been shown to be appropriate for burn studies (Bucky, et al., Plast. Reconstr. Surg., 93(7):1473-1480 (1994)). Further, the structural characteristics of the skin layers in rabbits and humans are similar. Three days prior to the operation, the backs of 10 New Zealand White Rabbits are depilated with a depilatory cream. Since the thickness of the skin is dependent upon the stage of the hair growth cycle, estimation of the hair growth pattern is carefully assessed. Immediately prior to infliction of the burn injury, the operation area is depilated a second time to achieve a smooth and hairless skin surface.

B. Operative Technique

Rabbits are sedated by intramuscular administration of ketamine (25 mg/kg BM) as described in the art (Knabl et al., Burns, 25:229-235 (1999)). A soldering iron with an adjustable aluminum contact stamp is used for infliction of the burn. The temperature of the stamp is set to 80° C. and continuously monitored. Bums are inflicted on the dorsal skin of the rabbits for approximately 14 seconds using only the weight of the stamp (approximately 85 g). The wounds are then immediately cooled with thermoelements which provide a consistent temperature of 10° C. for 30 minutes (Knabl, et al., supra).

To minimize the fact that different parts of the body with different skin thickness have differenct re-epithelialization and healing potentials, the same donor site on the animals is used. Therefore, any observed differences could be attributed to the treatment itself rather than to other variables. A Padget Electric Dermatome is used to harvest a 0.12 inch thick skin graft from the depilated thigh in all animals. The graft is carefully spread on the burn area. It is held in place either by gentle pressure from a well-padded dressing or by a few small stitches. The raw donor area is covered with a sterile non-adherent dressing for a 3-5 days to protect it from infection until full re-epithelialization is observed.

1×10⁹ pfu of AdVEGF, AdVEGF-C156S, AdVEGF-C, and AdLacZ are injected intradermally into the dorsal skin to the burn site of the rabbits. AdVEGF construction has been described previously (Makinen, et al., supra) and the AdVEGF-C156S, AdVEGF-C, AdLacZ vectors are constructed as described herein. As described in Example 2 above, reduction of edema and increase in skin perfusion at a burn wound site as a result of an increase in functional lymph nodes is assessed by following the accumulation of fluorescent dextran.

Additionally, healing is monitored by evaluating the cosmetic appearance of the skin graft. Normal graft color is similar to that of the recipient site. Surface temperature of the graft can be monitored using adhesive strips (for an accurate number) or the back of the hand (to provide a comparative assessment with the surrounding skin). Problems with arterial inflow are suggested when the graft is pale relative to the donor site and/or cool to the touch. Problems with venous outflow are suggested when the graft is congested and/or edematous. Color and appearance of congested grafts can vary depending on whether the congestion is mild or severe and ranges from a prominent pinkish hue to a dark bluish purple color.

C. Summary

The aforementioned model demonstrates the therapeutic potential of using VEGF-C and VEGF-C156S to preserve function of the lymphatic vessels and to improve healing and reduce edema and concomitant post-surgical complications in burn victims. Thus, the procedures and compositions described herein provide an important need in the art. Specifically, the reduction of edema or increase in perfusion at a burn site is accomplished, for example, by delivery of AdVEGF-C or AdVEGF-C156S to the site of the wound.

### EXAMPLE 6

### VEGF-C Therapy Following Mastectomy: An Animal Model

The following example describes a surgical procedure and delivery of VEGF-C156S and VEGF-C adenoviral vectors to breast tissue following a mastectomy procedure to improve healing.

A. Animals and Skin Preparation

Male guinea pigs of at least 3 months of age are used in the present model. The animals are anesthetized using ketamine and xyalzine as described (Eroglu et al., Eur. J. Surg. Onc., 22:137-139 (1996)). After shaving the anterior thoracic region, skin is disinfected with chlorohexidine solution.

B. Operative Technique

A mid-sternal incision is made from the jugular notch to xiphoid, and a skin flap is elevated from the sternum to axillary region. The flap is retracted laterally and the pectoralis major muscle is transected from is origin to insertion. Axillary dissection is performed with careful haemostasis by cautery and ligation if necessary. The wound is dried with sterile gauze after the operation.

1×10⁹ pfu of AdVEGF, AdVEGF-C156S, AdVEGF-C, and AdLacZ are injected intradermally into the site of incision of the guinea pigs. Adenoviral vector construction has been described above. As described in Examples 2 and 3 above, reduction of edema and increase in skin perfusion at a burn wound site as a result of an increase in functional lymph nodes is assessed by following the accumulation of fluorescent dextran.

Additionally, healing is monitored by evaluating the cosmetic appearance of the skin flap. Normal flap color is similar to that of the recipient site. Surface temperature of the flap can be monitored using adhesive strips (for an accurate number) or the back of the hand (to provide a comparative assessment with the surrounding skin). Problems with arterial inflow are suggested when the flap is pale relative to the donor site and/or cool to the touch. Problems with venous outflow are suggested when the flap is congested and/or edematous. Color and appearance of congested flaps can vary depending on whether the congestion is mild or severe and ranges from a prominent pinkish hue to a dark bluish purple color.

C. Summary

The aforementioned model demonstrates the therapeutic potential of using VEGF-C and VEGF-C156S to preserve function of the lymphatic vessels and to improve healing and reduce edema and concomitant post-surgical complications in mastectomy patients. Thus, the procedures and compositions described herein provide an important need in the art. Specifically, the reduction of edema or increase in perfusion at an incision site is accomplished, for example, by delivery of AdVEGF-C or AdVEGF-C156S to the site of the incision.

### EXAMPLE 7

### Naked VEGF-C Transgene Therapy

The procedures described in Examples 2-6 are repeated, with the following modifications. Instead of using an adenovirus vector for delivery of the VEGF-C transgene, a mammalian expression vector is constructed for direct gene transfer (of naked plasmid DNA). The VEGF-C coding sequence is operably linked to a suitable promoter, such as the CMV, K14, K5, K6, K16 or alpha 1(I) collagen promoter and preferably linked to a suitable polyadenylation sequence, such as the human growth hormone polyadenylation sequence. Exemplary VEGF-C vectors can be modeled from vectors that have been described in the literature to perform vector-free gene transfer for other growth factors, by substituting a VEGF-C coding sequence for a VEGF coding sequence. (See, e.g., Isner et al., Circulation, 91: 2687-2692 (1995); and Isner et al., Human Gene Therapy, 7: 989-1011 (1996), incorporated herein by reference) vector. A similar construct comprising a LacZ or Green fluorescent protein gene is used as a control.

### EXAMPLE 8

### VEGF-C Polypeptide Therapy

The procedures described in Examples 2-6 are repeated except, instead of treating the test animals with an adenovirus containing a VEGF-C transgene or lacZ control, the animals are treated with a composition comprising a VEGF-C polypeptide in a pharmaceutically acceptable carrier (e.g., isotonic saline with serum albumim), or with carrier solution alone as a control. Test animals receive either 10, 100, 250, 500, 1000, or 5000 μg of a VEGF-C polypeptide via intradermal injection, e.g., as described in Examples 2 and 3. A second group of animals additionally receive an injection of the VEGF-C polypeptide 7 days later. Accumulation of FITC-dextran can be monitored as described in Examples 2 and 3. Alternatively, the animals are sacrificed and histological examination performed as described in Examples 2 and 3. Repetition of the experiment using various sustained-release VEGF-C formulations and materials as described above is expected to further enhance the therapeutic efficacy of the VEGF-C polypeptide.

### EXAMPLE 9

### VEGF-D Polynucleotide and Polypeptide Therapy

The procedures described in the preceding examples are repeated using a composition comprising VEGF-D. Subjects are treated with a composition comprising a recombinant adenoviral VEGF-D (AdVEGF-D) or with a composition comprising a VEGF-D polypeptide.

### EXAMPLE 10

### Wound Healing Activity of Growth Factor Compositions

The procedures described in the preceding examples are repeated using a composition comprising either VEGF-C, VEGF-C156S, or VEGF-D in combination with one or more of the growth factors described herein. For example, a composition comprising a VEGF-C or VEGF-C156S polynucleotide or polypeptide may be administered to a subject in combination with one or more of the following: a VEGF, a VEGF-B, a VEGF-D, a VEGF-E, a P1FG, an Ang-1, an EGF, a PDGF-A, a PDGF-B, a PDGF-C, a PDGF-D, a TGF-β and/or an IGF polynucleotide or polypeptide.

Similarly, a composition comprising a VEGF-D polynucleotide or polypeptide may be administered to a subject in combination with one or more of the following: a VEGF, a VEGF-B, a VEGF-C, a VEGF-C156S, a VEGF-E, a P1FG, an Ang-1, an EGF, a PDGF-A, a PDGF-B, a PDGF-C, a PDGF-D, a TGF-β and/or an IGF polynucleotide or polypeptide.

### EXAMPLE 11

### Recombinant VEGF-C with Heparin Binding Property

The present Example describes the generation of chimeric VEGF-C molecules comprising an amino terminal VEGFR-3 binding domain of VEGF-C fused to a carboxy terminal heparin binding domain from VEGF. These molecules retain VEGFR-3 binding activity as shown by a cell survival assay and are expected to have an enhanced heparin binding activity as compared to native VEGF-C and enhanced angiogenic and/or lymphangiogenic properties.

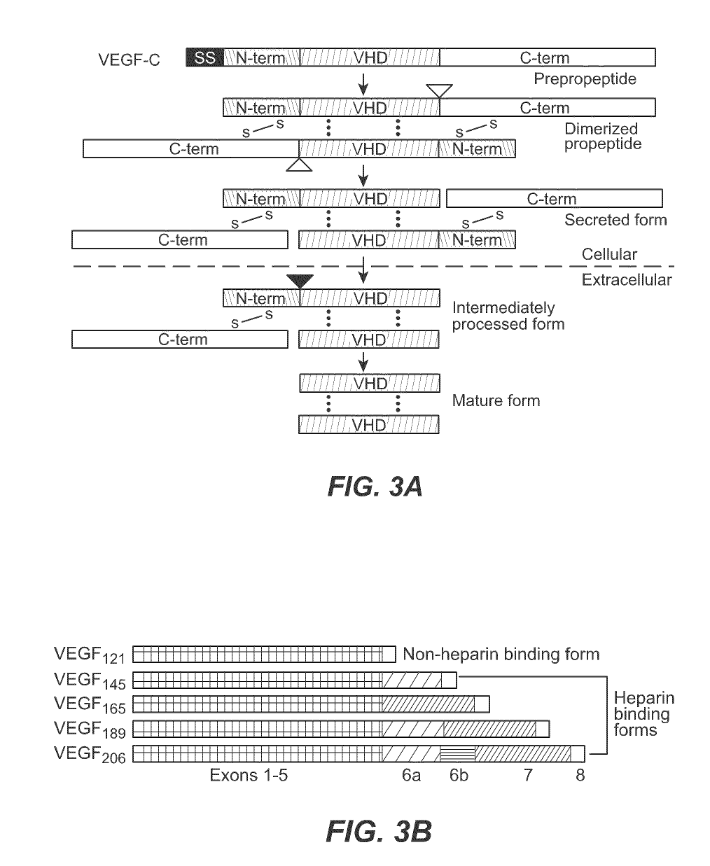

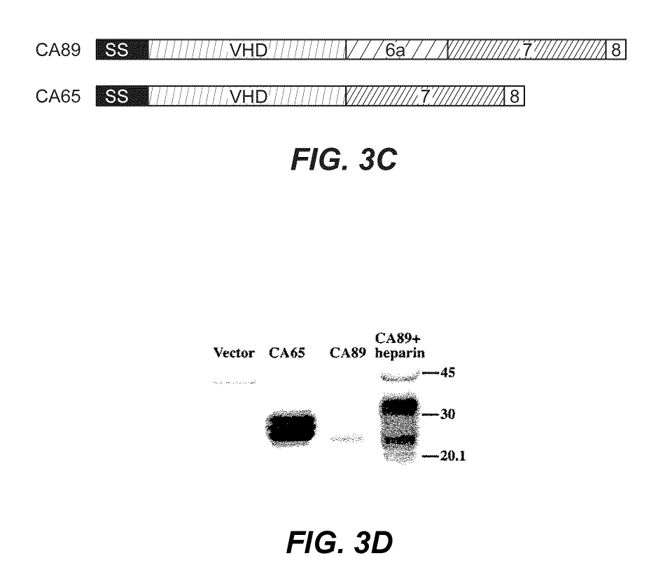

As described herein, the heparin-binding domain of VEGF or another heparin-binding growth factor may be fused to the growth factor domain of VEGF-C or VEGF-D to create heparin binding VEGFR-3 ligands. VEGF, which has potent angiogenic activity, includes a heparin binding domain. VEGF121 has potent angiogenic activity, but does not contain a heparin binding domain. The major forms of VEGF are VEGF121, VEGF145, VEGF165, VEGF189 and VEGF206, which result from alternative RNA splicing (FIG. 3B) (Ferrara and Davis-Smyth, Endocr Rev 18: 4-25, 1997). An important biological property that distinguishes these VEGF isoforms from each other is their different binding affinities to heparin and heparan sulfate. The four longer isoforms described above contain a heparin binding domain encoded by exon 6 and/or exon 7. The 21 amino acids encoded by exon 6 contain a heparin binding domain and also elements that enable binding to extracellular matrix (Poltorak et al., J. Biol. Chem. 272:7151-8, 1997). Molecules containing the cationic polypeptide sequence encoded by exon 7 (44 amino acids) are also heparin-binding and remain bound to the cell surface and the extracellular matrix. Recently, it has been shown that carboxymethyl benzylamide dextran, a heparin-like molecule, effectively inhibits the activity of VEGF165 by interfering with heparin binding to VEGF165 (Hamma-Kourbali et al., J Biol Chem., 276(43):39748-54, 2001). There is also other evidence that points to the importance of the heparin binding property of growth factors for their biological activities (Dougher et al., Growth Factors, 14: 257-68, 1997; Carmeliet et al., Nat Med 5: 495-502, 1999; Ruhrberg et al., Genes Dev 16 2684-98, 2002).

VEGF-C and VEGF-D do not have significant heparin binding activity (and, for the purposes of this invention, are not “heparin binding” as that term is used). In order to achieve maximum activation of VEGFR-2 and VEGFR-3 in vivo, and produce VEGF-C and/or VEGF-D molecules that are more potent in inducing angiogenesis and/or lymphangiogenesis, the inventors have produced or described chimeric molecules of VEGF-C and VEGF-D in which the VHD domain is fused or otherwise linked to a heparin binding domain. Methods and compositions for making and using these molecules are described in further detail herein below.

A. Chimeric VEGFR-3 Ligands that Bind Heparin

The present invention provides chimeric VEGFR-3 ligands of the formula X—B—Z or Z—B—X, where domain X binds Vascular Endothelial Growth Factor Receptor 3 (VEGFR-3) and domain Z comprises a heparin binding amino acid sequence. “Domain” B, which comprises a covalent attachment linking X to Z, and at its simplest, is nothing more than a peptide bond or other covalent bond Preferably, domain X comprises an amino acid sequence at least 90% identical to a prepro-VEGF-C amino acid sequence, a fragment of VEGF-C that possesses VEGFR3 binding activity, a prepro-VEGF-D amino acid sequence, or a fragment of VEGF-D that possesses VEGFR3 binding activity. These and other molecules that may serve as X are described in further detail herein.

The chimeric molecules are engineered to possess a heparin binding domain Z which preferably increases potency of the molecule as an inducer of angiogenesis and/or lymphangiogenesis, as compared to a similar VEGFR-3 ligand that lacks a heparin binding domain (such as wildtype VEGF-C or -D). This increase in potency may, for example, be due to an increase in the half-life of the chimeric molecule in vivo as compared to the unmodified VEGFR-3 ligand, or to better or more sustained localization in the bloodstream, lymph, or vessel tissues, or other tisses.

Domain X: a VEGFR-3 Binding Domain

The VEGFR-3 ligand binding domain of molecules can be any amino acid sequence that binds VEGFR-3, and confers VEGFR-3 binding to the molecules of the invention. For the purposes of the invention, VEGFR-3 binding means binding to the extracellular domain of human VEGFR-3 (Flt4 receptor tyrosine kinase) as described in U.S. Pat. No. 5,776,755, incorporated herein by reference. Molecules that have at least 10% of the binding affinity of fully-processed (mature) human VEGF-C or VEGF-D for VEGFR-3 are considered molecules that bind VEGFR-3.

Preferred VEGFR-3 binding domains share significant amino acid similarity to a naturally occurring vertebrate VEGF-C or VEGF-D, many of which have been described in the literature and others of which can be cloned from genomic DNA or cDNA libraries, and using PCR and/or standard hybridization techniques and using known VEGF-C or -D cDNAs as probes. For example, preferred molecules have at least 70% amino acid identity to a naturally occurring VEGF-C or -D protein or to a fragment thereof that binds VEGFR-3. Still more preferred are VEGFR-3 binding domains with at least 75%, 80%, 85%, 90%, 95%, 96%, 97%, 98%, or 99% amino acid sequence identity with the natural/wild type vertebrate VEGFR-3 ligand sequence. Descriptions herein of embodiments involving wild type sequences should be understood also to apply to variants sharing such amino acid similarity. It will be appreciated that conservative substitutions and/or substitutions based on sequence alignments with species homologues are less likely to diminish VEGFR-3 binding activity compared to the wild type reference sequence.

A very highly preferred wild type VEGFR-3 ligand for use as the VEGFR-3 binding domain is human prepro-VEGF-C and VEGFR-3 binding fragments thereof. Human VEGF-C polypeptides that may be used as domain X are described in WO 97/05250, WO 98/33917, WO 00/24412, and U.S. Pat. Nos. 6,221,839, 6,361,946, 6,645,933, 6,730,658 and 6,245,530, each of which is incorporated herein by reference in its entirety.

VEGF-C comprises a VHD that is approximately 30% identical at the amino acid level to VEGF. VEGF-C is originally expressed as a larger precursor protein, prepro-VEGF-C, having extensive amino- and carboxy-terminal peptide sequences flanking the VHD, with the C-terminal peptide containing tandemly repeated cysteine residues in a motif typical of Balbiani ring 3 protein. The nucleic acid and amino acid sequences of human prepro-VEGF-C are set forth in SEQ ID NO: 1 and SEQ ID NO:2, respectively. Prepro-VEGF-C undergoes extensive proteolytic maturation involving the successive cleavage of a signal peptide, the C-terminal pro-peptide, and the N-terminal pro-peptide, as described in Joukov et al. (EMBO J., 16:(13):3898 3911, 1997) and in the above-referenced patents. Secreted VEGF-C protein consists of a non-covalently linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. (Joukov et al., EMBO J., 16:(13):3898 3911, 1997). The entire text of U.S. Pat. No. 6,361,946 is incorporated herein by reference as providing a teaching of the sequence of the VEGF-C protein, gene and mutants thereof.

For treatment of humans, VEGF-C polypeptides with an amino acid sequence of a human VEGF-C are highly preferred, and polynucleotides comprising a nucleotide sequence of a human VEGF-C cDNA are highly preferred. By “human VEGF-C” is meant a polypeptide corresponding to a naturally occurring protein (prepro-protein, partially-processed protein, or fully-processed mature protein) encoded by any allele of the human VEGF-C gene, or a polypeptide comprising a biologically active fragment of a naturally-occurring mature protein. By way of example, a human VEGF-C comprises a continuous portion of the amino acid sequence set forth in SEQ ID NO: 2 sufficient to permit the polypeptide to bind VEGFR-3 in cells that express VEGFR-3. A polypeptide comprising amino acids 131-211 of SEQ ID NO: 2 is specifically contemplated. For example, polypeptides having an amino acid sequence comprising a continuous portion of SEQ ID NO: 2, the continuous portion having, as its amino terminus, an amino acid selected from the group consisting of positions 30-131 of SEQ ID NO: 2, and having, as its carboxyl terminus, an amino acid selected from the group consisting of positions 211-419 of SEQ ID NO: 2 are contemplated. As explained elsewhere herein in greater detail, VEGF-C biological activities, especially those mediated through VEGFR-2, increase upon processing of both an amino-terminal and carboxyl-terminal pro-peptide. Thus, an amino terminus selected from the group consisting of positions 102-131 of SEQ ID NO: 2 is preferred, and an amino terminus selected from the group consisting of positions 103-113 of SEQ ID NO: 2 is highly preferred. Likewise, a carboxyl terminus selected from the group consisting of positions 211-227 of SEQ ID NO: 2 is preferred. As stated above, the term “human VEGF-C” also is intended to encompass polypeptides encoded by allelic variants of the human VEGF-C characterized by the sequences set forth in SEQ ID NOs: 1 & 2.

Moreover, since the therapeutic VEGF-C is to be administered as recombinant VEGF-C or indirectly via somatic gene therapy, it is within the skill in the art (and an aspect of the invention) to make and use analogs of human VEGF-C (and polynucleotides that encode such analogs) wherein one or more amino acids have been added, deleted, or replaced with other amino acids, especially with conservative replacements, and wherein the VEGFR-3 binding activity has been retained. Analogs that retain VEGFR-3 binding biological activity are contemplated as VEGF-C polypeptides for use in the present invention. In a preferred embodiment, analogs having 1, 2, 3, 4, 5, 6, 7, 8, 9, 10, 11, 12, 13, 14, 15, 16, 17, 18, 19, 20, 21, 22, 23, 24, or 25 such modifications and that retain VEGFR-3 binding activity are contemplated as VEGF-C polypeptides for use in the present invention. Polynucleotides encoding such analogs are generated using conventional PCR, site-directed mutagenesis, and chemical synthesis techniques. Molecules that bind and stimulate phosphorylation of VEGFR-3 are preferred.

Conservative substitutions include the replacement of an amino acid by a residue having similar physicochemical properties, such as substituting one aliphatic residue (Ile, Val, Leu or Ala) for another, or substitution between basic residues Lys and Arg, acidic residues Glu and Asp, amide residues Gln and Asn, hydroxyl residues Ser and Tyr, or aromatic residues Phe and Tyr. Further information regarding making phenotypically silent amino acid exchanges may be found in Bowie et al., Science 247:1306 1310 (1990).

In another variation, the VEGR-3 binding domain has an amino acid sequence similar to or identical to a mutant VEGF-C, in which a single cysteine (at position 156 of the human prepro-VEGF-C sequence) is either substituted by another amino acid or deleted (SEQ ID NO: 6). Such VEGF-CΔCys156 (SEQ ID NO: 17) mutants, even when fully processed by removal of both pro-peptides, fail to bind VEGFR-2 but remain capable of binding and activating VEGFR-3. Such polypeptides are described in International Patent Publication No. WO 98/33917 and U.S. Pat. Nos. 6,130,071, and 6,361,946, each of which is incorporated herein by reference in its entirety, especially for their teachings of VEGF-C ΔCys156 molecules which may be used in producing chimeras of the present invention which comprise VEGF-C ΔCys156 as subunit X of the chimera.

Another highly preferred wild type VEGFR-3 ligand for use in constructing chimeric molecules of the invention is human VEGF-D. VEGF-D is initially expressed as a prepro-peptide that undergoes N-terminal and C-terminal proteolytic processing, and forms non-covalently linked dimers. VEGF-D stimulates mitogenic responses in endothelial cells in vitro. Exemplary human prepro-VEGF-D nucleic acid and amino acid sequences are set forth in SEQ ID NO:3 and SEQ ID NO:4, respectively. In addition, VEGF-D is described in greater detail in International Patent Publication No. WO 98/07832 and U.S. Pat. No. 6,235,713, each of which is incorporated herein by reference and describes VEGF-D polypeptides and variants thereof that are useful in producing the chimeras of the present invention. VEGF-D related molecules also are described in International Patent Publication Nos. WO 98/02543 and WO 97/12972, and U.S. Pat. No. 6,689,580, and U.S. patent application Ser. Nos. 09/219,345 and 09/847,524, all of which are incorporated by reference.

Isolation of a biologically active fragment of VEGF-D designated VEGF-DΔNΔC, is described in International Patent Publication No. WO 98/07832, incorporated herein by reference. VEGF-DΔNΔC consists of amino acid residues 93 to 201 of VEGF-D linked to the affinity tag peptide FLAG®. The prepro-VEGF-D polypeptide has a putative signal peptide of 21 amino acids and is apparently proteolytically processed in a manner analogous to the processing of prepro-VEGF-C. A “recombinantly matured” VEGF-D lacking residues 1-92 and 202-354 of SEQ ID NO: 4 retains the ability to activate receptors VEGFR-2 and VEGFR-3, and appears to associate as non-covalently linked dimers. Thus, preferred VEGF-D polynucleotides include those polynucleotides that comprise a nucleotide sequence encoding amino acids 93-201 of SEQ ID NO: 4, or comprising fragments thereof that retain VEGFR-3 and/or VEGFR-2 binding.

Moreover, since the therapeutic VEGF-D is to be administered as recombinant VEGF-D or indirectly via somatic gene therapy, it is within the skill in the art (and an aspect of the invention) to make and use analogs of human VEGF-D (and polynucleotides that encode such analogs) wherein one or more amino acids have been added, deleted, or replaced with other amino acids, especially with conservative replacements, and wherein the VEGFR-3 binding activity has been retained. Analogs that retain VEGFR-3 binding biological activity are contemplated as VEGF-D polypeptides for use in the present invention. In a preferred embodiment, analogs having 1, 2, 3, 4, 5, 6, 7, 8, 9, 10, 11, 12, 13, 14, 15, 16, 17, 18, 19, 20, 21, 22, 23, 24, or 25 such modifications and that retain VEGFR-3 binding activity are contemplated as VEGF-D polypeptides for use in the present invention. Polynucleotides encoding such analogs are generated using conventional PCR, site-directed mutagenesis, and chemical synthesis techniques. Molecules that bind and stimulate phosphorylation of VEGFR-3 are preferred.

Preferred fragments of VEGF-C or -D for use in making the chimeric molecules of the invention are continuous fragments that bind VEGFR-3. However, it has been demonstrated that VEGFR-3 binding can be achieved with molecules that incorporate discrete, discontinuous fragments of VEGF-C, fused, e.g., to fragments of VEGF-A or other amino acid sequences. Such chimeric VEGFR-3 ligands are described in U.S. patent application Ser. No. 09/795,006, filed Feb. 26, 2001, and International Patent Publication No. WO 01/62942, each of which is incorporated herein by reference in its entirety. The methods and compositions described in these documents may be used in the present invention to produce VEGF-C chimeras having a heparin binding domain. Moreover, the same teachings also apply to using continuous or discontinuous fragments of VEGF-D to make molecules that bind VEGFR-3.

In still another variation, the VEGFR-3 ligand sequence for use in making chimeras of the invention is itself a chimeric molecule comprised of VEGF-C and VEGF-D sequences. The foregoing documents describe methods for making such chimeras and confirming their VEGFR-3 binding activity.

In addition to binding VEGFR-3, the VEGFR-3 binding domain used to make molecules of the invention optionally also binds VEGFR-2. In addition, the molecule optionally binds VEGFR-1 and/or one or more neuropilin molecules.

Receptor binding assays for determining the binding of such chimeric molecules to one or more of these receptors are well-known in the art. Examples of such receptor binding assays are taught in e.g., U.S. patent application Ser. No. 09/795,006, and WO 01/62942, each incorporated herein by reference. (See, e.g., Example 3 of U.S. patent application Ser. No. 09/795,006, and WO 01/62942, which details binding assays of VEGF-C and related VEGF receptor ligands to soluble VEGF receptor Fc fusion proteins. Example 5 of those documents details analysis of receptor activation or inhibition by such ligands. Example 6 describes analyses of receptor binding affinities of such ligands.) In addition, Achen et al., Proc Natl Acad Sci USA 95:548 53 (1998), incorporated by reference in its entirety, teaches exemplary binding assays. The binding of the chimeric VEGF polypeptides having the formula X—B—Z to any one or more of VEGF receptors, VEGFR 1, VEGFR 2, and VEGFR 3, may be analyzed using such exemplary assays.

Domain Z: a Heparin Binding Domain

Domain Z of the chimeric X—B—Z molecules is any substance that possesses heparin binding activity and therefore confers such heparin binding activity to the chimeric polypeptide. Without being bound to any mechanisms of action, it is contemplated that the presence of a heparin binding domain on the growth factors facilitates the binding of the growth factors to heparin and allows the concentration of the growth factors in the extracellular matrix to increase the efficiency of binding of the growth factors to their respective cell surface receptors, thereby increasing the bioavailability of the growth factors at a given site.

VEGF-C and VEGF-D, like VEGF121, lack a heparin binding domain. However, it is known that VEGF145, VEGF165, VEGF189 and VEGF206, comprise heparin-binding domains (Keck et al., Arch. Bioch. Biophys., 344:103-113, 1997; Fairbrother et al., Structure 6:637-648, 1998). Exons 6 (21 amino acids) and 7 (44 amino acids) contain two independent heparin binding domains (Poltorak et al., Herz, 25:126-9, 2000). In preferred aspects of the present invention, subunit Z is a heparin binding domain encoded by exon 6, and/or exon 7 of VEGF. Subunit Z may further comprise the amino acids encoded by exon 8 of VEGF. The sequences of the various exons of VEGF are widely known and may be found at e.g., Genbank Accession numbers M63976-M63978, where M63976 is exon 6, M63977 is exon 7 and M63978 is exon 8.

As noted herein, the human VEGF-A gene is expressed as numerous isoforms, including VEGF145, VEGF165, VEGF189, and VEGF206. A human VEGF206 sequence obtained from the Swiss Prot database (accession no. P15692) is set forth below and in SEQ ID NO: 11:

1 mnfllswvhw slalllylhh akwsqaapma egggqnhhev vkfmdvyqrs ychpietlvd

61 ifqeypdeie yifkpscvpl mrcggccnde glecvptees nitmqimrik phqgqhigem

121 sflqhnkcec rpkkdrarqe kksvrgkgkg qkrkrkksry kswsvyvgar cclmpwslpg

181 phpcgpcser rkhlfvqdpq tckcsckntd srckarqlel nertcrcdkp rr

Amino acids 1-26 of this sequence represent the signal peptide and mature VEGF206 comprises amino acids 27-232. Referring to the same sequence, the signal peptide and amino acids 142-226 are absent in mature isoform VEGF121. The signal peptide and amino acids 166-226 are absent in mature isoform VEGF145. The signal peptide and amino acids 142-182 are absent in mature isoform VEGF165 (SEQ ID NO: 18). The signal peptide and amino acids 160-182 are absent in mature isoform VEGF183. The signal peptide and amino acids 166-182 are absent in mature isofrom VEGF189.

Referring to FIG. 3B and the foregoing sequence, amino acids 142-165 correspond to exon 6a (found in VEGF isoforms 145, 189, and 206); amino acids 166-182 correspond to exon 6b (found in isofrom 206 only); and amino acids 183-226 correspond to exon 7 (found in isoforms 165, 189, and 206).

Thus, referring again to the same sequence, the apparent heparin binding domain within VEGF145 corresponds to amino acids 142-165 or a fragment thereof. The apparent heparin binding domain of VEGF165 corresponds to amino acids 183-226 or a fragment thereof.

The apparent heparin binding domain(s) of VEGF189 (SEQ ID NO: 19) correspond to amino acids 142-165 joined directly to amino acids 183-226, or fragment(s)s thereof. The apparent heparin binding domain(s) of VEGF206 correspond to amino acids 142-226, or fragment(s) thereof.

In other embodiments, subunit Z may be derived from the heparin binding domains of other, non-VEGF growth factors. For example, subunit Z may be the heparin binding domain of VEGF-B. Makinen et al., (J. Biol. Chem., 274:21217-22, 1999), have described various isoforms of VEGF-B and have shown that the exon 6B encoded sequence of VEGF-B167 resembles the heparin and NRP1-binding domain encoded by exon 7 of VEGF165. Thus exon-6B of VEGF-B167 (or a heparin binding fragment thereof) may be used as the heparin binding subunit Z of the chimeric molecules of the present invention. The publication of Makinen et al., J. Biol. Chem., 274: 21217-22, 1999 provides a detailed description of the construction of the VEGF-B exon 6B-encoded sequence. Nucleotide and deduced amino acid sequences for VEGF-B are deposited in GenBank under Acc. No. U48801, incorporated herein by reference. Also incorporated herein by reference is Olofsson et al., J. Biol. Chem. 271 (32), 19310-19317 (1996), which describes the genomic organization of the mouse and human genes for VEGF-B, and its related Genbank entry at AF468110, which provides an exemplary genomic sequence of VEGF-B.

Mulloy et al., (Curr Opin Struct Biol. 11(5):623-8, 2001) describes properties from many heparin binding domain structures and identifies many heparin binding domain examples, and is incorporated herein by reference. Any such heparin binding domains may be used in the chimeric molecules of the present invention. In still further embodiments, subunit Z may comprise the heparin binding domain of P1GF-2 (see Hauser and Weich, Growth Factors, 9 259-68, 1993). Heparin binding domains from other growth factors also may be used in the present chimeric polypeptides, such as for example the heparin binding domain from EGF-like growth factor (Shin et al., J Pept Sci. 9(4):244-50, 2003); the heparin binding domain from insulin-like growth factor-binding protein (Shand et al., J Biol Chem. 278(20):17859-66, 2003), and the like. Other heparin binding domains that may be used herein include, but are not limited to, the pleiotrophin and amphoterin heparin binding domains (Matrix Biol. 19(5):377-87, 2000); CAP37 (Heinzelmann et al., Int J Surg Investig. 2(6):457-66, 2001); and the heparin-binding fragment of fibronectin (Yasuda et al., Arthritis Rheum. 48(5):1271-80, 2003).

Those of skill in the art are aware that heparin binding domains are present on numerous other proteins, including e.g., apolipoprotein E (SEQ ID NO: 20, residues 162-165, 229-236), fibronectin (SEQ ID NO: 21), amphoterin (SEQ ID NO: 22), follistatin (SEQ ID NO: 23), LPL (SEQ ID NO: 24), myeloperoxidase (SEQ ID NO: 25), other growth factors, and the like. Merely by way of example, the protein sequences of various heparin binding proteins found in Genbank include but are not limited to 1LR7_A; 1LR8_A; 1LR9_A; AAH05858 (FN1); NP_{—}000032 ( ); NP_{—}000177 (H Factor 1); NP_{—}001936 (dip theria toxin receptor); NP_{—}002328 (alpha-2-MRAP); NP_{—}005798 (proteoglycan 4); NP_{—}009014 ( ); NP_{—}032018; NP_{—}032511; NP_{—}034545; NP_{—}035047; NP_{—}037077; NP_{—}498403; NP_{—}604447; NP_{—}932158 ( ); NP_{—}990180; O15520; O35565; O46647; P01008; P02649; P02749; P02751; P04196; P04937; P05546; P05770; P06858; P07155; P07589; P08226; P10517; P11150; P11276; P11722_{—}1; P11722_{—}2; P15656; P15692; P17690; P18287; P18649; P18650; P20160; P23529; P26644; P27656; P30533; P33703; P35268; P47776; P49182; P49763; P51858; P51859; P55031; P61150; P61328; P61329; Q01339; Q01580; Q06186; Q11142; Q15303; Q28275; Q28377; Q28502; Q28640; Q28995; Q61092; Q61851; Q64268; Q7M2U7; Q8VHK7; Q91740; Q95LB0; Q99075; Q9GJU3; Q9WVG5; Q9Y5X9; XP_{—}357846; XP_{—}357859; XP_{—}358238; XP_{—}358249; 1304205A; 1AE5; 1B9Q_A; 1FNH_A; 1KMX_A; 1MKC_A; 1OKQ_A; A35969; A38432; A41178; A41914; A48991; AAA37542; AAA50562; AAA50563; AAA50564; AAA81780; AAB27481; AAB33125; AAC42069; AAD29416; B40080; C40862; I39383; IB9P_A; JC1409; JC1410; JC4168; JT0573; LPHUB; LPHUE; 018739; 019113; P11151; P11153; P11602; P12034; P13387; P41104; P48807; P49060; P49923; P55302; P70492; Q06000; Q06175; Q09118; Q11184; Q29524; Q91289; Q9CB42; Q9R1E9; S26049; S27162; S51242; XP_{—}134550; XP_{—}142078; XP_{—}145641; XP_{—}212881; XP_{—}213021; XP_{—}227645; XP_{—}232701; XP_{—}344685; XP_{—}344947; XP_{—}345821; XP_{—}346046; XP_{—}357159; XP_{—}357228; XP_{—}357258; XP_{—}358223.

In addition, the heparin binding domain may be one derived from any of these proteins. In exemplary embodiments heparin binding of the domain may be determined by e.g., heparin affinity chromatography. In alternative embodiments, the heparin binding domain may be assessed using methods described in U.S. Pat. No. 6,274,704. The heparin binding peptides described therein also may by useful.

Domain B: a Covalent Linkage between X and Z.

Within the chimeric molecules of the formula X—B—Z, the term B denotes a linkage, preferably a covalent linkage, between subunit X and subunit Z. In some embodiments, B simply denotes a covalent bond. For example, in a preferred embodiment, where X—B—Z comprises a single continuous polypeptide, B can denote an amide bond between the C-terminal amino acid of X and the N-terminal amino acid of Z, or between the C-terminal amino acid of Z and the N-terminal amino acid of X. Another way to describe such embodiments is by the simplified formulas X—Z or Z—X.

The linker may be an organic moiety constructed to contain an alkyl, aryl backbone and may contain an amide, ether, ester, hydrazone, disulphide linkage or any combination thereof. Linkages containing amino acid, ether and amide bound components will be stable under conditions of physiological pH, normally 7.4 in serum and 4-5 on uptake into cells (endosomes). Preferred linkages are linkages containing esters or hydrazones that are stable at serum pH but hydrolyse to release the drug when exposed to intracellular pH. Disulphide linkages are preferred because they are sensitive to reductive cleavage; amino acid linkers can be designed to be sensitive to cleavage by specific enzymes in the desired target organ. Exemplary linkers are set out in Blattler et al. Biochem. 24:1517-1524, 1985; King et al. Biochem. 25:5774-5779, 1986; Srinivasachar and Nevill, Biochem. 28:2501-2509, 1989.

In still other embodiments, entity B is a chemically, or otherwise, cleavable bond that, under appropriate conditions, allows the release of subunit X from subunit Z. For example domains X and Z can be covalently linked by one or more disulfide bridges linking cysteine residues of X and Z; or by mutual attachment to a distinct chemical entity, such as a carbohydrate moiety.

In particular embodiments, entity B comprises a peptide linker comprising from 1 to about 500 amino acids in length. Linkers of 4-50 amino acids are preferred, and 4-15 are highly preferred. Preferred linkers are joined N-terminally and C-terminally to domains X and Z so as to form a single continuous polypeptide. In certain embodiments, the peptide linker comprises a protease cleavage site selected from the group consisting of a Factor Xa cleavage site, an enterokinase cleavage site (New England Biolabs), a thrombin cleavage site, a TEV protease cleavage site (Life Technologies), and a PreScission cleavage site (Amersham Pharmacia Biotech). The presence of such cleavage sites between subunit X and subunit Z will allow for the efficient release of effective amounts of subunit X in a suitable proteolytic milieu.

Processing of VEGF-C and -D is believed to occur in part intracellularly, but processing of the amino terminal pro-peptide is believed to occur following secretion. Cleavage of this pro-peptide is apparently necessary for VEGFR-2-mediated activity. In one variation of the invention, subunit B comprises an amino acid sequence analogous to the VEGF-C or -D N-terminal pro-peptide processing site, to make subunits X and Z susceptible to cleavage by the same protease that process these N-terminal pro-pepticles in vivo.

For example, with respect to VEGF-C, propeptide cleavage can occur at about amino acids 102/103 of SEQ ID NO: 2, and a suitable subunit B optionally include about 3-30 amino acids upstream and downstream of this site. The analogous processing site of VEGF-D occurs between residues 92 and 93 of SEQ ID NO: 4.

The linker is optionally a heterologous protein polypeptide. The linker may affect whether the polypeptide(s) to which it is fused to is able to dimerize to each other or to another polypeptide. Other chemical linkers are possible, as the linker need not be in the form of a polypeptide. However, when the linker comprises a peptide, the binding construct (with linker) allows for expression as a single molecule. Linker may be chosen such that they are less likely to induce an allergic or antigenic reaction.

More than one linker may be used per molecule of X—B—Z or Z—B—X. The linker may be selected for optimal conformational (steric) freedom between the growth factor and heparin binding domains allow them to interact with binding partners. The linker may be linear such that X and Z are linked in series, or the linker may serve as a scaffold to which two or more X or Z binding units are attached. A linker may also have multiple branches. For example, using linkers disclosed in Tam, J. Immunol. Methods 196:17 (1996). X or Z domains may be attached to each other or to the linker scaffold via N-terminal amino groups, C-terminal carboxyl groups, side chains, chemically modified groups, side chains, or other means.

When comprising peptides, the linker may be designed to have sequences that permit desired characteristics. For example, the use of glycyl residues allow for a relatively large degree of conformational freedom, whereas a proline would tend to have the opposite effect. Peptide linkers may be chosen so that they achieve particular secondary and tertiary structures, e.g., alpha helices, beta sheets and beta barrels. Quarternary structure can also be utilized to create linkers that join two binding units together non-covalently. For example, fusing a protein domain with a hydrophobic face to each binding unit may permit the joining of the two binding units via the interaction between the hydrophobic interaction of the two molecules. In some embodiments, the linker may provide for polar interactions. For example, a leucine zipper domain of the proto-oncoproteins Myc and Max, respectively may be used. Luscher and Larsson, Ongogene 18:2955-2966 (1999). In some embodiments, the linker allows for the formation of a salt bridge or disulfide bond. Linkers may comprise non-naturally occurring amino acids, as well as naturally occurring amino acids that are not naturally incorporated into a polypeptide. In some embodiments, the linker comprises a coordination complex between a metal or other ions and various residues from the multiple peptides joined thereby.

Linear peptide linkers may have various lengths, and generally consist of at least one amino acid residue. In some embodiments the linker has from 1 to 10 residues. In some embodiments, the linker has from 1 to 50 residues. In some embodiments, the linker has from 1-100 residues. In some embodiments, the linker has from 1-1000 residues. In some embodiments the linker has 1-10,000 residues. In some embodiments the linker has more than 10,000 residues. In some embodiments, the linear peptide linker comprises residues with relatively inert side chains. Peptide linker amino acid residues need not be linked entirely or at all via alpha-carboxy and alpha-amino groups. That is, peptides may be linked via side chain groups of various residues. In some embodiments, a linker is used as is described in Liu et al. U.S. Pat. Appl. Pub. No. 2003/0064053.

As the chimeric polypeptides of the present invention have the ability to bind VEGFR-3 and have the ability to bind heparin, one method of obtaining a highly purified specimen would be to subject the chimeric polypeptides to two types of affinity purification. One affinity purification being based on VEGFR-3 binding property of the chimeric polypeptides and the second affinity purification being based on the heparin binding property of the chimeric polypeptides. Heparin-based affinity chromatography methods are well known. For example, one uses a commercially available heparin-Sepharose affinity chromatography system such as e.g., Heparin Sepharose™ 6 Fast Flow available from Amersham Biosciences (Piscataway, N.J.). Heparin Sepharose also is available from Pharmacia (Uppsula, Sweden). Other heparin affinity chromatography resins are available from Sigma Aldrich (St. Louis, Mo.). Exemplary protocols for purifying VEGF165 using Heparin-Sepharose CL6B affinity chromatography are presented by Ma et al., (Biomed Environ Sci. 14(4):302-11, 2001), Dougher et al., (Growth Factors, 14(4):257-68, 1997). Such methods could be used for the purification of the chimeric polypeptides of the present invention. Where these methods are used in conjunction with the FLT4 receptor-based affinity purification discussed above, the receptor-based affinity purification may be performed before or after the heparin binding affinity chromatography step.

Yet another affinity chromatography purification procedure that may be used to purify the chimeric polypeptides of the present invention employs immunoaffinity chromatography using antibodies specific for either the heparin binding domain of the chimeric polypeptides or more preferably antibodies specific for the domain X of the chimeric polypeptides. Antibodies specific for domain X would be any antibodies that are specific for VEGF-C, VEGF-D or chimeras of VEGF-D. In addition, purification of the chimeric polypeptides of the present invention may be achieved using methods for the purification of VEGF-C or VEGF-D that are described in U.S. Pat. No. 6,361,946 and WO 98/07832, respectively.

B. Nucleic Acids and Related Compositions.

The invention also embraces polynucleotides that encode the chimeric VEGF polypeptides discussed above and also polynucleotides that hybridize under moderately stringent or high stringency conditions to the complete non-coding strand, or complement, of such polynucleotides. Due to the well-known degeneracy of the universal genetic code, one can synthesize numerous polynucleotide sequences that encode each chimeric polypeptide of the present invention. All such polynucleotides are contemplated to be useful in the present application. Particularly preferred polynucleotides join a natural human VEGFR-3 receptor ligand cDNA sequence e.g., a sequence of SEQ ID NO:1 or SEQ ID NO:3, preferably a fragment thereof encoding a VEGFR-3 binding domain, with a natural human heparin binding domain encoding sequence. This genus of polynucleotides embraces polynucleotides that encode polypeptides with one or a few amino acid differences (additions, insertions, or deletions) relative to amino acid sequences specifically taught herein. Such changes are easily introduced by performing site directed mutagenesis, for example.

One genus of both polynucleotides of the invention and polypeptides encoded thereby can be defined by molecules with a first domain that hybridize under specified conditions to a VEGF-C or -D polynucleotide sequence and a second domain that hybridizes under the same conditions to naturally occurring human sequences that encode heparin binding domains taught herein.

Exemplary highly stringent hybridization conditions are as follows: hybridization at 65° C. for at least 12 hours in a hybridization solution comprising 5×SSPE, 5× Denhardt's, 0.5% SDS, and 2 mg sonicated non homologous DNA per 100 ml of hybridization solution; washing twice for 10 minutes at room temperature in a wash solution comprising 2×SSPE and 0.1% SDS; followed by washing once for 15 minutes at 65° C. with 2×SSPE and 0.1% SDS; followed by a final wash for 10 minutes at 65° C. with 0.1×SSPE and 0.1% SDS. Moderate stringency washes can be achieved by washing with 0.5×SSPE instead of 0.1×SSPE in the final 10 minute wash at 65° C. Low stringency washes can be achieved by using 1×SSPE for the 15 minute wash at 65° C., and omitting the final 10 minute wash. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook et al., (Eds.), Molecular Cloning: A Laboratory Manual, Cold Spring Harbor Laboratory Press: Cold Spring Harbor, New York (1989), pp. 9.47 to 9.51. For example, the invention provides a polynucleotide that comprises a nucleotide sequence that hybridizes under moderately stringent or high stringency hybridization conditions to any specific nucleotide sequence of the invention, and that encodes a chimeric polypeptide as described herein that binds at least one of the naturally occurring vascular endothelial growth factor or platelet derived growth factor receptors.

In a related embodiment, the invention provides a polynucleotide that comprises a nucleotide sequence that is at least 80%, 85%, 90%, 95%, 97%, 98%, or 99% identical to any specific nucleotide sequence of the invention, and that encodes a polypeptide that binds heparin and at least one of the naturally occurring vascular endothelial growth factor or platelet derived growth factor receptors.

C. Materials & Methods

Cloning: cDNAs encoding the fusion proteins comprised of the VEGF homology domain of VEGF-C and the C-terminus of VEGF (exon 6-8 encoded polypeptide fragment, referred to below as CA89, or exon 6-7 encoded fragment referred to below as CA65) were constructed by PCR amplification using the following primers: VEGF-CΔNΔC, 5′-ACATTGGTGTGCACCTCCAAGC-3′ (SEQ ID NO:12) and 5′-AATAATGGAATGAACTTGTCTGTAAAC-3′ (SEQ ID NO:13); VEGF C-terminal regions: 5′-AAATCAGTTCGAGGAAAGGGAAAG-3′ (SEQ ID NO:14) or 5′-CCCTGTGGGCCTTGCTCAGAG-3′ (SEQ ID NO:15), and 5′-CCATGCTCGAGAGTCTTTCCTGGTGAGAGATCTGG-3′ (SEQ ID NO:16). The PCR products were digested with HindIII (5′-HindIII/3′-blunt) or XhoI (5′-blunt-3′-XhoI), and cloned into the pEBS7 (Peterson and Legerski, Gene, 107 279-84, 1991) ) expression vector that had been digested with the same enzymes to create clones named pEBS7/CA89 and pEBS7/CA65. The inserts were also subcloned into pREP7 at HindIII/XhoI sites (pREP7/CA89 and pREP7/CA65.

Cell culture, transfection and immunoprecipitation: 293T and 293EBNA cells were maintained in DMEM medium supplemented with 2 mM L-glutamine, penicillin (100 U/ml), streptomycin (100 μg/ml), and 10% fetal bovine serum (Autogen Bioclear). BaF3 cells (Achen et al., Eur J Biochem., 267: 2505-15, 2000) were grown in DMEM as above with the addition of Zeocine (200 μg/ml) and the recombinant human VEGF-CdNdC (100 ng/ml).

293T cells were transfected with pEBS7/CA89, pEBS7/CA65 or the pEBS7 vector using liposomes (FuGENE 6, Roche). Cells transfected with pEBS7/CA89 were cultured with or without heparin (20 unit/ml). Transfected cells were cultured for 24 h, and were then metabolically labeled in methionine-free and cysteine-free modified Eagle medium supplemented with [35S]methionine/[35S]cysteine (Promix, Amersham Pharmacia Biotech) at 100 μCi/mL for 8 h. Conditioned medium was then harvested, cleared of particulate material by centrifugation, and incubated with polyclonal antibodies against VEGF-C [Joukov et al., EMBO J. 16:3898-911, 1997). The formed antigen-antibody complexes were bound to protein A Sepharose (Pharmacia Biotech), which were then washed twice with 0.5% bovine serum albumin/0.02% Tween 20 in phosphate-buffered saline (PBS) and once with PBS, and analysed in sodium dodecyl sulfate-polyacrilamide gel electrophoresis (SDS-PAGE) under reducing conditions.

293EBNA cells were transfected with pREP7/CA89, pREP7/CA65 or the pREP7 vector as described above. Cells transfected with pREP7/CA89 were cultured with or without heparin (20 unit/ml). The transfected cells were cultured for two days, and the supernatants were harvested for the assay of biological activity.

Bioassay for growth factor-mediated cell survival: Ba/F3 cells expressing a VEGFR-3/EpoR chimeric receptor (Achen et al., Eur J Biochem., 267: 2505-15, 2000) were seeded in 96-well plates at 15,000 cells/well in triplicates supplied with conditioned medium (0, 1, 5, 10 or 20 μl) from cell cultures transfected with pREP7/CA89, pREP7/CA65 or the pREP7 vector. Cell viability was measured by a colorimetric assay. MTT (3-(4,5-dimethylthiazol-2-yl)-2,5-diphenyl tetrazolium bromide (Sigma), 0.5 mg/ml) was added into each well and incubated for 4 h at 37° C. The reaction was terminated by adding 100 μl of lysis buffer (10% SDS, 10 mM HCl), and the resulting formazan products were solubilized overnight at 37° C. in a humid atmosphere. The absorbance at 540 nm was measured with a Multiscan microtiter plate reader (Labsystems).

E. Results & Discussion

The innate heparin binding property of certain growth factors has been implicated as important for their biological activities (Dougher et al., Growth Factors, 14: 257-68, 1997; Carmeliet et al., Nat Med 5: 495-502, 1999; Ruhrberg et al., Genes Dev 16 2684-98, 2002). VEGF (Poltorak et al., J. Biol. Chem. 272:7151-8, 1997; Gitay-Goren et al., J Biol Chem 271: 5519-23, 1996), VEGF-B167 (Makinen et al., J. Biol. Chem., 274:21217-22, 1999), and P1GF-2 (Hauser and Weich, Growth Factors, 9 259-68, 1993) all possess significant heparin binding activity, but VEGF-C and VEGF-D do not. Both of these latter molecules have been shown to induce lymphangiogenesis in transgenic mice and in other in vivo models (Jeltsch et al., Science 276:1423-5, 1997; Oh et al., Dev Biol 188: 96-109, 1997; Veikkola et al., EMBO J 20: 1223-31, 2001). Although recombinant proteins of mature forms of VEGF-C and VEGF-D are believed to exert angiogenic activity via VEGFR-2 (Cao et al. Proc Natl Acad Sci USA 95: 14389-94, 1998; Marconcini et al., Proc Natl Acad Sci USA 96: 9671-6, 1999), mature forms of VEGF-C delivered by other means such as adenoviral vectors have so far induced weak angiogenic activity in mice. These data suggest that the concentration of the protein present may not be sufficient, or that the half-life of the mature form of VEGF-C protein may be too short to induce a potent angiogenic effect. Maximum activation of VEGFR-2 in vivo may also require the ligand to have the property of heparin binding, as suggested for VEGF (Dougher et al., Growth Factors, 14: 257-68, 1997).

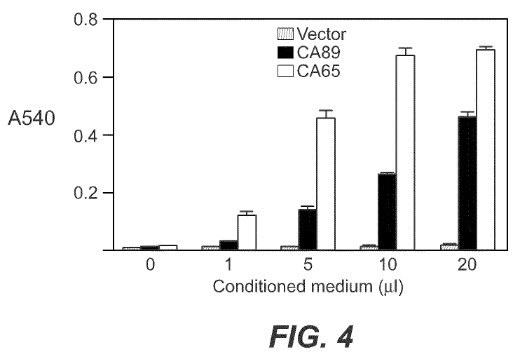

To investigate the effects of introducing a heparin binding activity on the angiogenic and lymphangiogenic effects of VEGF-C, plasmids encoding chimeric proteins comprised of the signal sequence and the VEGF homology domain (VHD) of VEGF-C, and VEGF exon 6-8 or exon 7-8 encoded sequences (FIG. 3C) were constructed. Expression of the chimeric VEGF-C proteins by the transfected cells was confirmed by immunoprecipitation with polyclonal antibodies against VEGF-C (FIG. 3D). CA65 was secreted and released into the supernatant, but CA89 was not released into the supernatant unless heparin was included in the culture medium (FIG. 3D), indicating that it apparently binds to cell surface heparan sulfates similar to what has been described for VEGF189. VEGFR-3-mediated biological activity of the chimeric proteins (CA89 and CA65) was demonstrated by a bioassay using Ba/F3 cells expressing a chimeric VEGFR-3/erythropoietin (Epo) receptor (Ba/F3/VEGFR-3). Conditioned medium from both 293EBNA/CA89 and 293EBNA/CA65 cells were shown to induce survival and proliferation of the IL-3 dependent Ba/F3/VEGFR-3 cells in the absence of the recombinant IL-3 protein (FIG. 4). The effect was detectable even with 1 ml of the conditioned medium added.

Lymphatic vessels typically accompany blood vessels. The chimeric molecules of the present invention may allow efficient localization of growth factors expressed in a given tissue, without the danger of obtaining aberrant side effects in other sites/organs. Secondly, the heparin binding forms would allow a growth factor gradient to be established for vessel sprouting. Further, given the teachings described herein, the chimeric polypeptides of the present invention which are heparin binding factors give enhanced lymphangiogenic and/or angiogenic effects, as their three dimensional diffusion is replaced by two-dimensional diffusion in the plane of the cell surface heparin matrix, which leads to a more concentrated form of the growth factor available for the high-affinity signal transducing receptors. Furthermore, heparin binding forms of VEGF containing the VEGF exon 7-encoded sequence can also bind to neuropilins, which have important roles in the development of the cardiovascular system and the lymphatic system. Consequently, the putative neuropilin-1 binding property of the chimeric polypeptides of the invention could direct VEGF-C towards more efficient stimulation of angiogenesis.

### EXAMPLE 12

### VEGF-C Fused to Heparin-Binding Domain Has Increased Lymphangiogenic Activity

The present example further demonstrates that chimeric VEGF-C molecules containing a heparin binding domain have increased lymphangiogenic activity in comparison with the VEGF-CΔNΔC form. The enhancement of the biological activity may result from an increased bioavailability of the protein, or increased receptor binding via binding to NP-1 or NP-2. Without being bound to any theory of mechanism of action, it is possible that the presence of the heparin binding domain facilitates a two-dimensional diffusion of the heparin-domain-containing chimeric VEGF-C molecules such that the chimeric molecules become distributed in the plane of the cell surface heparin sulphate matrix, which leads to a more concentrated form of the growth factor presented and available for the high-affinity signal-transducing receptors. Furthermore, the heparin binding forms may allow a growth factor gradient to be established for vessel sprouting.

A. Materials and Methods

The methods described in the previous Examples are incorporated into the present Example by reference. The studies described in the present example also employed the following additional experimental protocols.

Production and in vivo delivery of CA89 and CA65 by viral vectors. The AAV vector psub-CAG-WPRE was cloned by substituting the CMV promoter fragment of psub-CMV-WPRE (Paterna et al., Gene Ther., 7(15):1304-1311, 2000) with the CMV-chicken beta-actin insert (Niwa et al., Gene, 108(2):193-199, 1991). The cDNAs encoding CA89 and CA65 were cloned as blunt-end fragments into the psub-CAG-WPRE plasmid, and the recombinant AAV viruses (AAV.CA89 and AAV.CA65, AAV serotype 2) were produced as previously described in Karkkainen et al., Proc. Natl. Acad. Sci. USA, 98(22):12677-12682 (2001). The cDNAs encoding CA89 and CA65 were also cloned into the pAdBglII vector (AdCA89 and AdCA65), and recombinant adenoviruses were produced as described in Laitinen et al., Hum. Gene Ther., 9(10):1481-1486, 1998. NCI-H460-LNM35 cells (Kozaki et al., Cancer Res., 60(9):2535-2540, 2000) were used for expression analysis. These cells were maintained in RPMI1640 medium with supplements as above and were infected with AAV.CAG.VEGFR-3-Ig viruses (MOI 2000), or adenoviruses (MOI 50). Expression of the recombinant proteins were examined by metabolic labeling, immunoprecipitation followed by SDS-PAGE analysis as described above.

Adenoviruses (AdCA89 or AdCA65, approximately 3×108 pfu), or AAV viruses (AAV.CA89, AAV.CA65 or AAV.EGFP, approximately 1×1010 viral particles) were injected subcutaneously into mouse ears. Tissues were collected for analysis after two weeks with adenoviruses and three weeks with AAV viruses for histological analysis.

Fluorescent microlymphography. The functional lymphatic network in the ears was visualized by fluorescent microlymphography using dextran conjugated with fluorescein isothiocyanate (molecular weight: 2000 kDa, Sigma) that was injected intradermally into the ears. The lymphatic vessels were examined using a dissection microscope (LEICA MZFLIII).

Immunohistochemistry. For whole mount staining, tissues were fixed in 4% paraformaldehyde (PFA), blocked with 3% milk in PBS, and incubated with polyclonal antibodies against LYVE-1 (Prevo et al., J. Biol. Chem., 276(22):19420-12930, 2001) and PECAM-1 (PharMingen) overnight at 4° C. Alexa594 and Alexa488 conjugated secondary antibodies (Molecular Probes) were used for staining, and samples were then mounted with Vectashield (Vector Laboratories) and analysed with a Zeiss LSM510 confocal microscope. For staining of tissue sections, tissues were fixed in 4% PFA overnight at 4° C. and paraffin sections (6 μm) were immunostained with anti-LYVE-1 and monoclonal antibodies against PECAM-1.

B. Results and Discussion

As discussed in Example 11, the heparin binding property of growth factors is important in the biological activities of those factors that bind heparin. The data shown in Example 11 demonstrated that the presence of a heparin binding domain have an enhanced heparin binding activity as compared to native VEGF-C and enhanced angiogenic and/or lymphangiogenic properties. The following discussion further corroborates those findings.

Enhancement of receptor binding activity of recombinantly processed VEGF-C by addition of heparin binding domain. Analysis of the receptor binding profiles of the chimeric molecules showed that, similar to VEGF-CΔNΔC, both CA89 and CA65 bound to VEGFR-2, VEGFR-3, but not VEGFR-1 (FIG. 5B). Heparin binding forms of VEGF, containing the VEGF exon 7-encoded sequence, have been shown to bind to neuropilins, which have important roles in the development of the cardiovascular and lymphatic systems (Soker et al., J. Biol. Chem., 271(10):5761-5767, 1996; Neufeld et al., Trends Cardiovasc. Med., 12(1):13-19, 2002). In agreement with these data, both CA89 and CA65 bound to NP-1 and NP-2, whereas VEGF-CΔNΔC had a weak binding activity to NP-2 but did not bind to NP-1 (FIG. 5A).

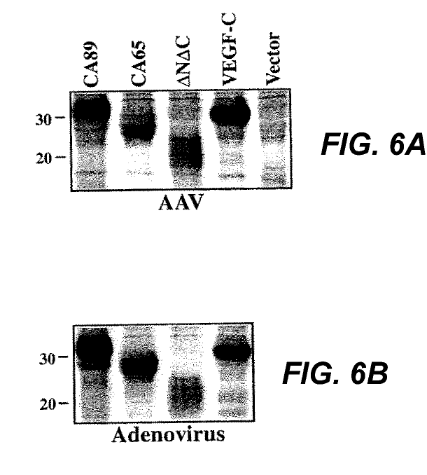

Lymphangiogenic activity of VEGF-CΔNΔC is enhanced by heparin/neuropilin binding domain. To further characterize the biological functions of the chimeric proteins in vivo, the cDNAs encoding CA89 and CA65 were cloned into the pAdBglII vector (AdCA89 and AdCA65) for the generation of recombinant adenoviruses. Recombinant AAV (AAV.CA89 and AAV.CA65, serotype 2) were also produced to study the effect of long-term expression of the chimeric molecules. Shown in FIG. 6 is the analysis of polypeptides produced via the AAV (FIG. 6A) and adenoviral (FIG. 6B) expression of CA89, CA65, VEGF-C and the VEGF-CΔNΔC.

For analysis of their in vivo vascular effects, adenoviruses encoding CA89, CA65, and VEGF-CΔNΔC were injected subcutaneously into the ears of nude mice. AdVEGF-C (full length/“prepro-VEGF-C”) and AdLacZ viruses were used as positive and negative controls. Tissues were collected for whole mount immunostaining of lymphatic vessels (LYVE-1 antigen) and blood vessels (PECAM-1) within two weeks. Both CA89 and CA65 were shown to induce strong lymphangiogenesis in comparison with the LacZ control. While CA89 exerted a localized effect around the virus injection site, CA65 induced a widespread effect in a fashion similar to the full-length VEGF-C. This is in agreement with the differential distribution of the two chimeric molecules between pericellular matrix and fluid phases in culture. VEGF-CΔNΔC induced only a weak lymphangiogenic effect with some lymphatic sprouting from the pre-existing lymphatic vessels. There was no angiogenic effect observed with the heparin binding chimeric molecules, VEGF-CΔNΔC or full length VEGF-C in comparison with the control.

Both CA89 and CA65 delivered by the recombinant AAV viruses also induced strong lymphangiogenesis when compared with the control involving AAV.EGFP. However, the effects observed with AAV vectors were seen only around the ear muscles, as AAV viruses mainly transduce muscle and neurons (Daly, Methods Mol. Biol., 246:157-165, 2004). The lymphatic vessels grew along the muscle fibers that were transduced with AAV.EGFP. These data indicate that by use of a vector/tissue-specific promoter and a heparin-binding growth factor, one can achieve a more defined localization of growth factor expression in a given tissue, and therefore minimize the danger of obtaining aberrant side effects from other sites.

However, analysis by microlymphography showed that the lymphatic vessels generated in the mice receiving CA89 or CA65 via viral vectors were leaky compared with the control. Similar findings have been reported for vessels generated with full-length VEGF-C, and the results suggest that a combination of CA89 or CA65 with other molecules such as Ang-1 is necessary for the optimal induction of functional lymphatic vessels.

In histological sections from the AAV.CA89 treated mice, many LYVE-1-positive vessel-like structures were observed in regions close to cartilage where the ear muscles are located, whereas only a few lymphatic vessels were found in corresponding sections from the control mice. PECAM-1, a panendothelial marker for blood and lymphatic vessels, also detected more vessels in the sections from the AAV.CA89 treated mice. Similarly, many LYVE-1-positive vessel-like structures, often in clusters close to the cartilage, were found in the AAV.CA65 treated mice. In contrast, fewer lymphatic vessels were observed in the control mice.

In summary, these experiments show the lymphangiogenic and/or angiogenic properties of VEGF-C short form in the presence and absence of a heparin binding property. Chimeric proteins made of the signal sequence and the VEGF homology domain (VHD) of VEGF-C, and the C-terminal domain of VEGF165 or VEGF189 isoforms containing heparin and neuropilinl binding sequences (named CA89 and CA65) were studied. CA65 was secreted and released into the supernatant, but CA89 was only released if heparin was included in the culture medium. Analysis of the receptor binding profiles of the chimeric molecules showed that they retained VEGFR-2 and VEGFR-3 binding and activation and in addition also bind to NP-1, whereas the VEGF-C short form did not retain these binding activities. In vivo expression of the chimeric proteins delivered via adenoviral or associated virus vectors demonstrated that they induced strong lymphangiogenesis in a mouse ear model, whereas angiogenic activity was not observed. The enhanced lymphangiogenic activity may result from the increase of its bioavailability and/or neuropilin binding property.

### EXAMPLE 13

### Use of Heparin Binding VEGFR-3 Ligands and Polynucleotides to Improve Healing

The procedures of preceding examples are repeated using heparin binding VEGFR-3 ligands described herein. Improved healing using such ligands (or polynucleotides encoding such ligands) provides an indication that such ligands are useful to improve wound healing.

While the present invention has been described in terms of specific embodiments, it is understood that variations and modifications will occur to those in the art, all of which are intended as aspects of the present invention. Accordingly, only such limitations as appear in the claims should be placed on the invention.

1. A method of improving the healing of a skin graft or skin flap to underlying tissue of a mammalian subject, comprising:
contacting skin graft or skin flap tissue or underlying tissue with a composition comprising a VEGF-D polypeptide,
wherein the VEGF-D polypeptide is present in said composition in an amount effective to reduce edema or increase perfusion at the skin graft or skin flap, thereby improving the healing of the skin graft or skin flap.

2. A method according to claim 1 wherein said mammalian subject is human.

3. A method according to claim 2, further comprising a step of attaching the skin graft or skin flap tissue to the underlying tissue.

4. A method according to claim 3 wherein the contacting precedes the attaching.

5. A method according to claim 3 wherein contacting is subsequent to the attaching.

6. (canceled)

7. A method according to claim 3 wherein the skin graft or skin flap is attached in a breast augmentation, breast reduction, mastopexy, or gynecomastia procedure.

8. A method according to claim 3 wherein the skin graft or skin flap is attached in a cosmetic surgery procedure.

9. A method according to claim 7, wherein the procedure is a facial cosmetic procedure selected from the group consisting of rhytidectomy, browlift, otoplasty, blepharoplasty, rhinoplasty, facial implant, and hair replacement therapy.

10. A method according to claim 3, wherein the skin graft or skin flap is attached in an abdominoplasty (abdominal lipectomy) or liposuction procedure.

11. A method according to claim 3, wherein the skin graft or skin flap is attached in a reconstructive surgery.

12. A method according to claim 10, wherein the reconstructive surgery corrects a congenital defect selected from the group consisting of birthmark, cleft palate, cleft lip, syndactyly, urogenital and anorectal malformations, craniofacial birth defects, ear and nasal deformitites, and vaginal agenesis

13. A method according to claim 10, wherein the reconstructive surgery corrects a defect from an injury, infection, or disease.

14. A method according to claim 12, wherein the injury is a burn.

15. A method according to claim 12, wherein the disease is skin cancer.

16. A method according to claim 12, wherein the reconstructive surgery is breast reconstruction following mastectomy or injury.

17. A method according to claim 3, wherein the skin graft is a split thickness, full thickness, or composite graft.

18. A method according to claim 3, wherein the skin flap is selected from the group consisting of a local flap, a regional flap, musculocutaneous flap, an osteomyocutaneous flap and soft tissue flap.

19. A method according to claim 1, wherein the contacting step comprises injecting the composition intradermally or subdermally.

20. A method according to claim 18, wherein the contacting comprises injection into the dermis of the skin graft or skin flap.

21. A method according to claim 1, wherein the contacting step comprises topical application of the composition to the skin graft or skin flap.

22-42. (canceled)

43. A method according to claim 1, further comprising contacting the skin graft or skin flap with an angiogenic growth factor.

44. A method according to claim 21, wherein the angiogenic growth factor is substantially free of vascular permeability increasing activity.

45. (canceled)

46. A method according to claim 2, wherein said administering comprises at least one intravascular injection of said composition.

47. A method according to claim 2 wherein said administering comprises a patch- or dressing-mediated transfer of said composition to the skin graft or skin flap.

48. A method according to claim 2, wherein the mammalian subject is diabetic.

49. A patch comprising a pad material having an upper surface and lower surface, an adhesive on the lower surface, and a therapeutic composition,
wherein the composition comprises a VEGF-D polypeptide.

50. A surgical suturing thread impregnated with a composition,
wherein the composition comprises a VEGF-D polypeptide.

51-53. (canceled)

54. A method according to claim 1 wherein the healing agent comprises a VEGF-D polypeptide.

55. A method according to claim 28 wherein said VEGF-D polypeptide comprises the amino acid sequence set forth in SEQ ID NO: 4 or a fragment thereof that binds VEGFR-3.
